(19) United States
(12) Patent Application Publication
(10) Pub. No.: US 2025/0297214 A1
(43) Pub. Date: Sep. 25, 2025

(54) METHODS FOR ISOLATING NEURAL STEM AND PROGENITOR CELLS FROM THE DEVELOPING HUMAN BRAIN

(71) Applicant: The Board of Trustees of the Leland Stanford Junior University, Stanford, CA (US)

(72) Inventors: Daniel Dan Liu, Redwood City, CA (US); Joy Q. He, Redwood City, CA (US); Rahul Sinha, Los Altos, CA (US); Nobuko Uchida, Palo Alto, CA (US); Irving L. Weissman, Stanford, CA (US)

(21) Appl. No.: 18/863,954

(22) PCT Filed: May 19, 2023
(86) PCT No.: PCT/US2023/022897

§ 371 Date: Nov. 7, 2024

Related U.S. Application Data
(60) Provisional application No. 63/344,461, filed on May 20, 2022.
(60) Provisional application No. 63/434,381, filed on Dec. 21, 2022.

(51) Int. Cl. C12N 5/0797; A61K 35/30; A61P 25/00; C12N 5/00; G01N 15/10; G01N 15/14; G01N 15/149; G01N 33/533; G01N 33/569

(52) U.S. Cl. CPC C12N 5/0623; A61K 35/30; A61P 25/00; C12N 5/0081; G01N 15/1459; G01N 15/149; G01N 33/533; G01N 33/56966; G01N 2015/1006; G01N 2333/705

(57) ABSTRACT

Methods for identifying, isolating and enriching neural stem and progenitor cells (NSPC) such as ventricular radial glia, outer radial glia, astrocytes, pre-oligodendrocyte precursor cells, oligodendrocyte precursor cells, oligodendrocytes, early excitatory neurons, late excitatory neurons, bipotent glial progenitors, and inhibitory neurons are provided. These methods find use in transplantation, to eliminate specific cell subsets, for experimental evaluation, as a source of lineage and cell-specific products, and the like, for example for use in treating human disorders of the central nervous system (CNS).

### CROSS-REFERENCE

This application claims benefit of U.S. Provisional Patent Application No. 63/344,461, filed May 20, 2022, and U.S. Provisional Patent Application No. 63/434,381, filed Dec. 21, 2022 which application are incorporated herein by reference in its entirety.

### BACKGROUND OF THE INVENTION

The human brain boasts an intricate architecture built from billions of cells of diverse identities. Yet, this extraordinary complexity arises during development from a relatively uniform neuroepithelium. In the developing cerebral cortex, radial glia (RG) are thought to serve as neural stem cells (NSCs) that self-renew and give rise to progressively more lineage-restricted progenitors, ultimately generating three major neural lineages: neurons, oligodendrocytes, and astrocytes. Recently, single-cell technologies have offered unprecedented spatial and temporal resolution of the transcriptomic diversity of neural stem and progenitor cells (NSPCs) throughout development, resulting in detailed atlases of human NSPCs. However, stem cells are defined not by their transcriptomic signatures, but rather their function, specifically with respect to their self-renewal and differentiation potential. The essence of stem cell biology thus lies in the ability to prospectively isolate pure populations of cells for functional characterization-warranting reliable tissue dissociation protocols and cell-surface markers for purification and assessment of their in vivo properties of self-renewal and differentiation. This is especially true in human tissues, for which researchers are not afforded the usual tools of genetic lineage tracing. Without such capabilities, questions regarding neural stem and progenitor mechanisms, that could include hierarchical organization of distinct intermediate progenitor states that are progressively restricted in their lineage output potential, have remained difficult to explore.

It has been previously shown that CD133⁺CD24^{-/lo} cells isolated from 16-20-gestational week-old (GW16-20) human brains using FACS are highly enriched in NSCs, with an appreciable fraction (1 in 23; range: 1 in 15 to 1 in 34) giving rise to clonally derived neurospheres in culture. The neurospheres, which consist of undifferentiated cells, expand for many passages, can differentiate into neurons and glia in vitro, and when transplanted into the brains of neonatal immunodeficient mice, engraft, migrate, proliferate, and differentiate in a site-appropriate manner. Cultured neurospheres are further capable of rescuing disease phenotypes in animal models of various central nervous system disorders. and have been extensively investigated for their clinical applications in regenerative medicine.

However, despite new NSC cell-surface markers that have since been identified in rodents, the precise identity and purity of cells sorted using combinations of these markers have not been rigorously characterized, and significant heterogeneity likely exists among the isolated cell populations. For example, radial glia exist in several distinct types: ventricular radial glia (vRG) which reside in the ventricular zone (VZ) and maintain both apical and basal processes, and outer radial glia (ORG) which reside in the outer subventricular zone (OSVZ) and maintain only their basal processes. However, a paucity of isolation methods for these rare, more nuanced cell types has made their functional characterization challenging.

Provided herein are methods for identifying, isolating and enriching NSPC populations of high purity based on cell-surface marker immunophenotypes.

### SUMMARY OF THE INVENTION

Methods for identifying, isolating and enriching neural stem and progenitor cells (NSPC) for the ventricular radial glia, outer radial glia, astrocytes, pre-oligodendrocyte precursor cells, oligodendrocyte precursor cells, oligodendrocytes, early excitatory neurons, late excitatory neurons, inhibitory neuron; and bipotent glial progenitor lineages are provided. These methods find use in transplantation, for experimental evaluation, as a source of lineage and cell-specific products, and the like, for example for use in treating human disorders of the central nervous system (CNS).

As described in the present disclosure, distinct NSPC populations were identified, isolated and enriched from brain tissue samples based on specific cell surface markers, which set of specific cell surface markers may be referred to herein as an “immunophenotype”. NSPCs were labeled with an antibody panel and were selected based on such an immunophenotype. The purity of isolated NSPC populations were assessed by correlating their transcriptomes with their immunophenotype. Isolated NSPCs are shown to retain the functional characteristics of their respective populations. These NSPC populations can be cultured to form neurospheres, which can be maintained indefinitely in culture. Among the neural cells, CD24⁻THY1^{-/lo} cells were enriched for radial glia, which robustly engrafted, migrated. and differentiated into all three neural lineages in the mouse brain. THY1^{hi} cells marked unipotent oligodendrocyte precursors committed to an oligodendroglial fate, and CD24⁺THY1^{-/lo} cells marked committed excitatory and inhibitory neuronal lineages. A transcriptomically-distinct THY1^{hi}EGFR^{hi}PDGFRA⁻ bipotent glial progenitor that is lineage-restricted to astrocytes and oligodendrocytes, but not neurons.

Transplantation of isolated NSPCs showed robust site-specific migration and engraftment, giving rise to the respective lineages following in vivo differentiation. Three major neural compartments are identified: CD24-THY1^{-/lo} NSCS, THY1^{hi} OPCs, and CD24⁺THY1^{-/lo} neuron precursors, with further immunophenotypic heterogeneity present within each main group. Whereas NSCs maintain multilineage potential, OPCs, astrocytes, and neuron precursors are heavily skewed if not outright committed to their specific lineage.

Using the immunophenotypes disclosed herein, a variety of NSPCs are identified, isolated and enriched for, including, without limitation, the lineages for ventricular radial glia (vRG), outer radial glia (oRG), astrocytes (AC). pre-oligodendrocyte precursor cells (pre-OPC), oligodendrocyte precursor cells (OPC), oligodendrocytes (OL), early excitatory neurons (early ExN), late excitatory neurons (late ExN), inhibitory neurons (InN), etc. In one embodiment the NSPC is a bipotent glial progenitor (GP), giving rise exclusively to oligodendrocytes and astrocytes.

In the methods of the invention, NSPCs are identified, isolated, and prospectively enriched from a brain tissue sample, based on their immunophenotype. Prior to isolation of the NSPCs, single cells are first dissociated from brain tissue samples. Single cell dissociation can be performed using a combination of mechanical and enzymatic dissociation. NSPCs can be identified, isolated and enriched from any suitable brain tissue sample using the methods disclosed herein. In some embodiments, the brain tissue sample is from a mouse. In some embodiments, the brain tissue sample is from a human. When the brain tissue sample is from a human, the brain tissue sample may be from a specific time point in brain development. For example, the human brain tissue sample may be from a fetal brain or an adult brain. In some embodiments, the brain tissue sample is from a fetal brain at the gestational age of 14-28 weeks. In a preferred embodiment, the brain tissue sample is from a fetal brain with the gestation age of 16-18 weeks.

Following single cell dissociation from brain tissue samples, single cells are contacted with an antibody panel comprising one or more of an anti-PROM1 (CD133), anti-CD24, anti-THY1(CD90), anti-CXCR4, anti-EGFR, anti-PDGFRA, anti-CD45, anti-PECAM1 (CD31), anti-CD34, anti-ENG (CD105), and anti-GYPA (CD235a) antibody, thereby producing labeled single cells. An antibody panel may comprise two, three, four, five, six, seven, eight, nine, ten or all eleven antibodies in the panel. In some embodiments, one or more of the antibodies of the antibody panel is conjugated to a fluorochrome. Labeled single cells are then selected based on binding to the antibody or antibodies. In some embodiments, labeled single cells are selected using fluorescence activated cell sorting (FACS).

The immunophenotype of the NSPCs define which population of NSPC the cells belong to. Ventricular radial glia can be defined by the immunophenotype CD24^{-/lo} THY1^{-/lo} EGFR^{hi}. Outer radial glia can be defined by the immunophenotype CD24^{-/lo} THY1^{-/lo} EGFR−. Astrocytes can be defined by the immunophenotype CD24^{-/lo} THY1^{-/lo} EGFR+ CXCR4+. Pre-oligodendrocyte precursor cells can be defined by the immunophenotype THY1hi EGFR+ PDGFRA+. Oligodendrocyte precursor cells can be defined by the immunophenotype THY1hi EGFR− PDGFRA+. Oligodendrocytes can be defined by the immunophenotype THY1hi EGFR− PDGFRA−. Early excitatory neurons may be defined by the immunophenotype CD24+ THY1^{/lo} CXCR4− EGFR−. Late excitatory neurons may be defined by the immunophenotype CD24+ THY1^{-lo} CXCR4− EGFR+. Inhibitory neurons may be defined by the immunophenotype CD24+ THY1^{-/lo} CXCR4+ EGFR−. A bipotent glial progenitor that is lineage-restricted to astrocytes and oligodendrocytes may be defined by the immunophenotype THY1^{hi}EGFR^{hi}PDGFRA⁻.

In some embodiments, ventricular radial glia are further defined by expression of transcripts of one or more of SOX2, GFAP, VIM, CRYAB or FBXO32. In some embodiments, outer radial glia are further defined by expression of transcripts of one or more of SOX2, GFAP, VIM, HOPX or LIFR.

In some embodiments, astrocytes are further defined by expression of transcripts of one or more of SOX2, GFAP, VIM, PAX3 or EDNRB.

In some embodiments, pre-OPCs are further defined by expression of transcripts of one or more of OLIG1, OLIG2, SOX10, EGFR, MKI67, or PCNA.

In some embodiments, OPCs are further defined by expression of transcripts of one or more of OLIG1, OLIG2, SOX10, PDGFRA, or PCDH15.

In some embodiments, OLs are further defined by expression of transcripts of one or more of OLIG1, OLIG2, SOX10, MYRF, or MBP. In some embodiments, early ExNs are further defined by expression of transcripts of one or more of DCX, SOX4, SOX11, or NEUROD2.

In some embodiments, late ExNs are further defined by expression of transcripts of one or more of DCX, SOX4, SOX11, or SATB2.

In some embodiments, InNs are further defined by expression of transcripts of one or more of DCX, SOX4, or SOX11.

In some embodiments, glial bipotent progenitors (GP) express ETV4 (ETS Variant Transcription Factor 4) as a GP-specific transcription factor. GPs may also be characterized as A2B5⁺.

In some embodiments, the transcripts of NSCPs are measured using single cell RNA sequencing (scRNA-seq).

NSCPs populations isolated using the methods disclosed herein are of high purity. For example, the NSPC populations can have a purity of at least about 75%, 80%, 85%, 90%, 95%, or a purity of greater than 95%.

In some embodiments, NSCPs identified, isolated and enriched using the above-mentioned methods have a greater likelihood to produce neurospheres relative to NSCPs isolated using different methods. For instance, NSCPs isolated using the above-mentioned methods may have a greater than 1.5, 2, 3, 4, 5, 6, 7, 8, 9, 10, or greater than 10 times higher likelihood of forming neurospheres relative to NSPCs isolated using conventional methods. In some embodiments, among the isolated NSCPs using the methods disclosed herein, NSCPs comprising an immunophenotype comprising either EGFRhi or EGFR+ have a higher likelihood of producing neurospheres. For example, isolated NSPCs comprising an immunophenotype comprising either EGFRhi or EGFR+ cells have a greater than 1.5, 2, 3, 4, 5, 6, 7, 8, 9, 10, or greater than 10 times higher likelihood of forming neurospheres relative to NSPCs isolated that do not comprise an immunophenotype comprising either EGFRh or EGFR.

In some aspects of the invention, methods are provided for treating an individual in need of cell transplantation therapy in the CNS. Successful transplantation to date only comes from early CNS stem and progenitor cells. In some embodiments, the cell transplantation therapy is neuron transplantation therapy, meaning replacement or addition of neurons derived from early CNS stem and/or progenitor cells. In some such embodiments, the subject is contacted with a composition of NSPCs isolated using the methods of the invention. In some embodiments, the individual has a CNS condition. In some embodiments, the CNS condition is a neurodegenerative disease, a neuropsychiatric disorder, a channelopathy, a lysosomal storage disorder, an autoimmune disease of the CNS, a cerebral infarction, a stroke, or a spinal cord injury. In other aspects a known CNS cell type can become a brain cancer stem cell, and in these cases the therapy is designed to eliminate just that cell type.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1D. Prospective isolation of NSPCs from the developing human brain. A. Tissue processing and experimental workflow for isolation and characterization of NSPCs via transcriptomic and functional methods. B. Gating scheme for isolation of distinct NSPC populations using FACS, based on varying cell-surface expression of THY1 (CD90), CD24, EGFR, PDGFRA, and CXCR4. Events are pre-gated on live single cells and negative for non-neural lineage markers PECAM1 (CD31), CD34, PTPRC (CD45), ENG (CD105), and GYPA (CD235a). C. Single cell RNA sequencing of index-sorted NSPCs using Smart-seq3. Plot showing PAGA-embedded Leiden clusters with annotated cell type identities based on expressed transcripts of known genes. D. Index-sort data was used to map the sequenced single cells to their original immunophenotype with respect to cell-surface CD24 and THY1 expression levels.

FIGS. 2A-2H. CD24⁻THY1^{-/lo} expression identifies NSCs. A. Gating scheme for CD24⁻THY1^{-/lo}. NSC compartment. B. Index-sort data was used to map the transcriptomically-identified vRG (red), oRG (orange), and AC (brown) clusters to their original immunophenotype. C. Plot showing the quantification of neurosphere initiation frequency of each CD24⁻THY1^{-/lo} subset based on in vitro limiting dilution assays. n=5-11 donors per population, mean +standard deviation (S.D.). D. Right: immunofluorescence (IF) images of CD24⁻THY1^{-/lo} subsets showing DCX (green) and GFAP (red) expression after 5 days of in vitro differentiation post-sort. Left: bar-graph showing quantification of percent DCX and GFAP' cells in images similar those shown in right panel. Error bars show standard deviation. (E-G) Confocal IF images of mouse brains engrafted with CD24^{-THY}1^{-/lo} human NSCs. 40 μm thick sections were stained with anti-human GFAP (E) or human cytoplasmic antigen-specific STEM121 antibody (F, G), in addition to species cross-reactive antibodies against SOX2, OLIG2, or NeuN and MAP2 (E, F, G, respectively). Imaged regions: medulla (Med), third ventricle (3V), hypothalamus (HY), olfactory bulb (OB), cerebellum (CB), corpus callosum (cc), optic chiasm (och), and subventricular zone (SVZ). Scale bar 25 μm. (H) Visualization of CD24⁻THY1⁻EGFR⁻ (top) and CD24⁻THY1^{-/lo}EGFR^{hi} (middle) NSCs engrafted into the mouse brain. Each dot represents an engrafted GFAP+ (orange) or an OLIG2+ (green) human cell. Bottom: bar graph showing quantification of orange GFAP+and green OLIG2+cells per mm3 in brain sections similar to those shown in the upper two panels. n=4 quantified brains per population; mean±S.D.

FIGS. 3A-3G. THY1^{hi} expression identifies OPCs. A. Gating scheme for THY1^{hi} OPC compartment. B. Index-sort data was used to map the transcriptomically-identified pre-OPC (light green), OPC (green), and OL (dark green) clusters to their original immunophenotype. C. (left) Quantification of processes on EGFR⁺PDGFRA⁺ and EGFR⁻PDGFRA⁺ cells in fetal human cerebral cortex (18 gestational weeks, GW18). (right) Confocal IF images of GW18 fetal human cerebral cortex stained for EGFR (green), PDGFRA (red), and DAPI (blue). Empty arrowheads to EGFR⁺PDGFRA⁺ cells; solid arrowheads point to EGFR⁻PDGFRA⁺ cells. D. Plot showing the quantification of neurosphere initiation frequency of each THY1^{hi} subset based on limiting dilution assays. n=4-7 donors per population, mean±S.D. E. IF images of the bulk-sorted THY1^{hi} subsets that were cultured in the absence of growth factors for 4 days, and subsequently stained with anti-O4 (green) antibody and DAPI (blue) marking the nuclei, along with quantification of percent O4⁺ cells in images similar to those in the left panel. Mean±S.D. F. Visualization of THY1^{hi} subsets engrafted into the mouse brain. Each dot represents an engrafted GFAP⁺ (orange) or OLIG2⁺ (green) human cell. G. Confocal IF images of mouse brains engrafted with THY1^{hi} human OPCs. 40 μm thick sections were stained with human cytoplasmic antigen specific STEM121 antibody (green) and OLIG2 (red). Imaged regions: corpus callosum (cc), cerebellum (CB), dentate gyrus (DG), and midbrain (MB). Scale bar 25 μm.

FIGS. 4A-4K. Identification of a bipotent glial progenitor. A. mRNA expression matrix showing astrocyte and oligodendrocyte marker genes in transcriptomic cell clusters. B. PAGA pseudotime analysis of expressed transcripts along the maturation trajectory from ventricular and outer radial glia (vRG, oRG) to glial progenitors (GP) to oligodendrocyte precursor cells (OPC) to oligodendrocytes (OL). C. Confocal immunofluorescence (IF) images of fetal human brain sections (18 gestational weeks; GW18) from the cortex. 14 μm thick sections were stained with antibodies against GFAP (green) and OLIG2 (red). Scale bar 50 μm. D. Confocal IF images of GW18 fetal human cerebral cortex, stained with antibodies against GFAP (green), OLIG2 (red), ETV4 (cyan, top), and HOPX (cyan, bottom). Scale bar 10 μm. E. Anatomical distribution of GFAP⁺OLIG2⁺ cells in GW18 fetal human cortex across the ventricular/subventricular zone (VZ/SVZ), outer subventricular zone (OSVZ), intermediate zone/subplate (IZ/SP), and cortical plate (CP). F. Index-sort data was used to map the transcriptomically-identified glial progenitors to their original immunophenotype. Glial progenitors were found to be enriched in the THY1^{hi}EGFR^{hi}PDGFRA⁻ gate. G. Experimental strategy for clonal differentiation assay. H. IF images of cells after differentiation stained with DAPI (blue) and antibodies against GFAP (red), O4 (green), and DCX (cyan). Clonal neurospheres were derived from single THY1^{hi}EGFR^{hi}PDGFRA⁻ cells, the cells from clonal neurospheres were dissociated then subjected to differentiation conditions. I. Quantification of lineage output of clonal neurospheres derived from THY1^{hi}EGFR^{hi}PDGFRA⁻, THY1^{hi}EGFR^{mid}PDGFRA⁻, THY1^{hi}EGFR⁺PDGFRA⁺, or CD24-THY1^{/lo} cells. Each column represents a distinct clonal neurosphere. Differentiated cells were classified based on their expression of GFAP, DCX, or OLIG2. J. Confocal IF images of mouse brains engrafted with THY1^{hi}EGFR^{hi}PDGFRA⁻ putative glial progenitors. 40 μm thick sections were stained with antibodies against human GFAP (left, green), SOX2 (left, red), human cytoplasmic antigen (right, green) and OLIG2 (right, red). Imaged regions: medulla (Med) and midbrain (MB). Scale bar 50 μm. K. Visualization of THY1^{hi}EGFR^{hi}PDGFRA⁻ cells engrafted into the mouse brain. Each dot represents an engrafted GFAP⁺ (orange) or OLIG2⁺ (green) human cell.

FIGS. 5A-5E. CD24⁺THY1^{-/lo} expression identifies neuron precursors. A. Gating scheme for CD24⁺THY1^{/lo} neuron compartment. B. Index-sort data was used to map the transcriptomically-identified early ExN (light blue), late ExN (dark blue), and InN (purple) clusters to their original immunophenotype. C. PAGA pseudotime analysis of expressed transcripts along the neuronal maturation trajectory. D. Left: IF images of cultured CD24⁺THY1^{-lo} CXCR4⁻ cells stained with anti-DCX (red) and anti-MAP2 (green) antibodies. Right: bar-graph showing the quantification of percent DCX⁺, MAP2⁺, GFAP⁺, and OLIG2⁺ cells in images similar to those shown in the left panel. E. IF images of cultured CD24^{-THY}1^{-lo}CXCR4⁻ cells stained with anti-SYN1 (red) and anti-MAP2 (green) antibodies.

FIGS. 6A-6B. NSPC surface markers are conserved across diverse brain regions. A. Dissected regions of fetal human brain. B. Sorting strategy for neural stem and progenitor cell types. Each gate has been assigned a letter from a-i. Stacked bar graphs show the composition of transcriptomically-defined cell types purified from each gate, for each brain region. (a) CD24⁻THY1^{-/lo}, (b) CD24⁺THY1^{-/lo}, (c) THY1^{hi}, (d) CD24⁺THY1^{-/lo}/CXCR4⁻, (e) CD24⁺THY1^{-/lo}CXCR4⁺, (f) THY1^{hi}EGFR^{hi}PDGFRA⁻, (g) THY1^{hi}EGFR⁺PDGFRA⁺, (h) THY1^{hi}EGFR⁻PDGFRA⁺, (i) THY1^{hu}EGFR⁻PDGFRA⁻.

FIG. 7. Surfaceomic profiling of NSPC types. Surface marker expression within each immunophenotypic population. presented as the percent of cells within the parent gate staining positive for that marker. Each bar graph represents one marker, with each bar representing an immunophenotypic population as denoted in the legend.

FIG. 8. Graphical summary of NSPC cell types and their corresponding surface marker expression identified in this study.

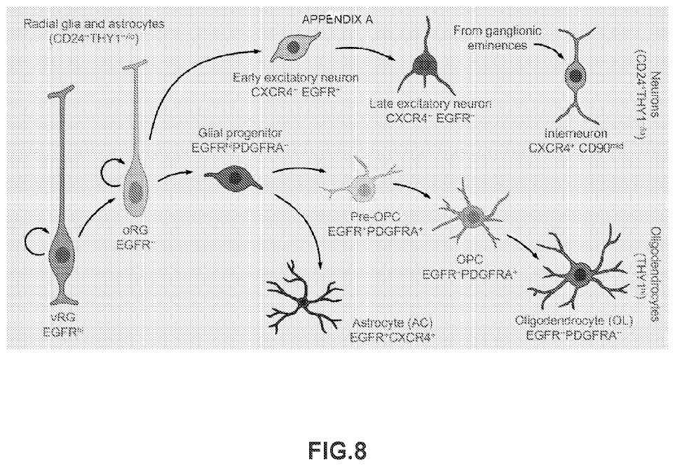

FIGS. 9A-9E. A. Pre-gating scheme for live, lineage negative single cells. Debris was removed based on forward and side scatter area, followed by two-step doublet discrimination using forward and side scatter height and width. Dead cells were removed based on staining for propidium iodide, and non-neural lineages were removed based on staining for fluorophore-conjugated antibodies against PECAM1 (CD31), CD34, PTPRC (CD45), ENG (CD105), or GYPA (CD235a). B. Dot plot showing marker gene expression for transcriptomically-defined clusters from single cell RNA sequencing. C. Scatter plots showing correspondence between RNA and protein expression in single cells. Cells are colored by their transcriptomically-defined clusters. D. Index-sort data was used to map the sequenced single cells to their original immunophenotype with respect to CD133 and CD24 cell-surface expression. E. Violin plot showing CD133 cell-surface protein expression as measured by flow cytometry in each transcriptomically-defined cluster as shown in FIG. 1C.

FIGS. 10A-10G. A. Flow cytometric analysis of cell-surface EGFR and LIFR expression within CD24⁻THY1^{/lo} (orange) gate. B. Confocal immunofluorescence (IF) imaging of fetal human cerebral cortex (18 gestational weeks) stained for DAPI (blue), GFAP (red), EGFR (green), and CRYAB (magenta). The boxed region shown at higher resolution on the right. Labelled regions: ventricular zone (VZ), subventricular zone (SVZ), outer subventricular zone (OSVZ), cortical plate (CP). Scale bar 500 μm. C. Computationally predicted cell cycle status in CD24⁻THY1^{-/lo} subsets. D. Intracellular staining for GFAP, SOX1, SOX2, and PAX6 in CD24⁺THY1^{-/lo} (blue), THY1^{hi} (green), and CD24⁻THY1^{-/lo} (orange) gates, besides corresponding isotype controls (grey). E. Experimental strategy for in vitro clonal neurosphere differentiation assays. Single CD24⁻THY1^{-/lo} cells that were either EGFR⁻ or EGFR⁺ were sorted into a well and cultured for 4 weeks to generate clonal neurospheres, which were then dissociated and subjected to differentiation. F. IF images of clonally derived cells stained with either (left) anti-O4 (green), or (right) anti-DCX (green) and anti-GFAP (red) antibodies. G. IF images of sorted CD24⁻THY1^{-/lo}EGFR⁺CXCR4⁺ cells subject to 5 days of in vitro differentiation and then stained for GFAP (red) and AQP4 (green).

FIGS. 11A-11F. A. Index-sort analysis of vRG and oRG cells found within the heterogeneous THY1^{hi}EGFR⁺PDGFRA⁻ gate. B. PAGA pseudotime analysis showing the oligodendrocyte maturation trajectory. C. PAGA pseudotime distribution within each THY1^{hi} subset. D. Computationally predicted cell cycle status in THY1^{hi} subsets. E. Confocal immunofluorescence (IF) images of fetal human cerebral cortex (18 gestational weeks) stained for EGFR (green), PDGFRA (red), and DAPI (blue). F. Quantification of O4⁺ cells among bulk sorted CD24⁻THY1^{-/lo} and CD24⁺THY1^{-/lo} populations cultured for 5 days. G. Flow cytometric analysis of sorted THY1^{hi}EGFR⁺PDGFRA⁺ and THY1^{hi}EGFR⁺PDGFRA⁻ cells that were cultured in vitro for 5 days. Each row represents a separate donor.

FIGS. 12A-12D. A. mRNA expression matrix showing glial progenitor (GP) marker genes and their expression in other cell types. B. Immunofluorescent (IF) imaging of sorted CD24⁻THY1^{-/lo} cells after being subjected to 5 days of in vitro differentiation. Cells were stained for DAPI (blue), GFAP (red), and OLIG2 (green). Scale bar 50 μm. C. Plot showing UMAP representation of single cell RNA sequencing of fetal NSPCs, with GP cluster highlighted (left), and expression plots for ETV4, ADM. and METTL7B (right). D. Flow cytometric analysis of A2B5 expression on NSPC subsets.

FIGS. 13A-13C. A. Single cell RNA sequencing of index-sorted NSPCs from dissected brain regions using Smart-seq3. Plot showing UMAP-embedded Leiden clusters with annotated cell type identities based on expressed transcripts of known genes. B. Stacked bar graph showing distribution of each transcriptomic cell type across brain regions. C. Plot showing the quantification of neurosphere initiation frequency of CD24⁻THY1^{-/lo}EGFR⁻ (orange) and CD24⁻THY1^{-/lo}EGFR^{hi} (red) cells from each brain region based on limiting dilution assays. Numbers underneath denote the reciprocal of the neurosphere initiation frequency. Error bars represent 95% confidence interval.

FIGS. 14A-14F. A. Dissected regions of fetal human brain. B. Representative flow cytometry plots showing the expression of THY1 and CD24 in cells derived from the subventricular zone or the thalamus. Events are pre-gated on live, lineage (blood and vessel) negative single cells. C. Frequency of THY1^{hi}, CD24⁻THY1^{-/lo}, and CD24⁺THY1^{-/lo} cells among live, lineage negative single cells. D. Frequency of EGFR⁻, EGFR^{mid}, EGFR^{hi}, and EGFR⁺CXCR4⁺ cells among CD24⁻THY1^{-/lo} cells. E. Frequency of EGFR^{hi}PDGFRA⁻, EGFR⁺PDGFRA⁺, EGFR⁻PDGFRA⁺, and EGFR⁻PDGFRA⁻ cells among THY1^{hi} cells. F. Frequency of CXCR4⁻EGFR⁻, CXCR4⁻EGFR⁺, and CXCR4⁺ cells among CD24⁺THY1^{-/lo} cells. Numbers shown below each bar denotes percentage of parent population.

FIGS. 15A-15C. A. Heat map showing the percentage of cells within each immunophenotypic population (rows) that stained positive for each surface marker (columns). B. Scatter plots showing correlation between RNA and surface antigen expression, plotted for each cell type. Each dot represents a marker, with the y-axis denoting the percentage of cells within the immunophenotypic gate staining positive for surface antigen, and the x-axis denoting the percentage of cells within the transcriptomic cluster expressing transcript for the marker. C. Scatter plot of markers, arranged on the x-axis by the percentage of cells staining positive for surface antigen (averaged across all 10 populations), and on the y-axis by the absolute difference between RNA and protein percent positivity, summed across all 10 populations.

### DETAILED DESCRIPTION OF THE EMBODIMENTS

Before the present methods and compositions are described, it is to be understood that this invention is not limited to particular method or composition described, as such may, of course, vary. It is also to be understood that the terminology used herein is for the purpose of describing particular embodiments only. and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either. neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad. Stratagene, Invitrogen, Sigma-Aldrich, and ClonTech.

By “proliferate” it is meant to divide by mitosis, i.e. undergo mitosis. An “expanded population” is a population of cells that has proliferated, i.e. undergone mitosis, such that the expanded population has an increase in cell number, that is, a greater number of cells, than the population at the outset.

The term “explant” refers to a portion of an organ or tissue therein taken from the body and cultured in an artificial medium. Cells that are grown “ex vivo” are cells that are taken from the body in this manner, temporarily cultured in vitro, and returned to the body.

The term “primary culture” denotes a mixed cell population of cells from an organ or tissue within an organ. The word “primary” takes its usual meaning in the art of tissue culture. Primary tissue, or primary tissue derived cells refers to cells that have not been expanded or maintained in culture.

The term “tissue” refers to a group or layer of similarly specialized cells which together perform certain special functions.

The term “organ” refers to two or more adjacent layers of tissue, which layers of tissue maintain some form of cell-cell and/or cell-matrix interaction to form a microarchitecture.

The terms “individual,” “subject,” “host,” and “patient,” are used interchangeably herein and refer to any mammalian subject for whom diagnosis, treatment, or therapy is desired, particularly humans.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it: (b) inhibiting the disease symptom, i.e., arresting its development; or (c) relieving the disease symptom, i.e., causing regression of the disease or symptom.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning. The methods of the invention also include the use of factor combinations that consist, or consist essentially of the desired factors.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

The term “progenitor cell” as used herein refers to a cell population that generates at least one differentiated progenitor, and may give rise to multiple lineages. Progenitor cells may self-renew, i.e. when the cells undergo mitosis, they produce at least one daughter cell that is a progenitor cell, although typically the self-renewal is of limited duration relative to stem cells. The cells are not pluripotent, that is, they are not capable of giving rise to cells of other organs in vivo.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). A “stem cell” is a cell characterized by the ability of self-renewal through mitotic cell division and the potential to differentiate into a tissue or an organ. Among mammalian stem cells, embryonic and somatic stem cells may be distinguished. Pluripotent stem cells, which include embryonic stem cells, embryonic germ cells and induced pluripotent cells, can contribute to tissues of a prenatal, postnatal or adult organism.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The term “neural stem and progenitor cells” or “NSPCs” encompasses cells of neural stem, neural, astrocyte and oligodendrocyte lineage. These include a variety of cells such as glial bipotent progenitor (BP), ventricular radial glia (vRG), outer radial glia (oRG), astrocytes (AC), pre-oligodendrocyte precursor cells (pre-OPC), oligodendrocyte precursor cells (OPC), oligodendrocytes (OL), early excitatory neurons (early ExN), late excitatory neurons (late ExN), inhibitory neurons (InN), or intermediate progenitor cells (IPC).

A “neurosphere”, as used herein, refers to is an aggregate or cluster of cells such as NSPCs. Neurospheres generated from a specific NSCP population comprises primarily NSPCs from the population isolated, e.g. neurospheres generated from vRG comprises primarily vRGs. Methods of neurosphere generation are well known in such as those disclosed in disclosed in Weiss et al., U.S. Pat. No. 5,750,376 and Weiss et al., U.S. Pat. No. 5,851,832, Marshal et al. (Methods Mol Biol. 2008; 438:135-150), each of which is specifically incorporated by reference herein.

For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers. etc.

The cell population may be used immediately. Alternatively, the cell population may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

A cell transplant, as used herein, is the transplantation of one or more cells into a recipient body, usually for the purpose of augmenting function of an organ or tissue in the recipient. As used herein, a recipient is an individual to whom tissue or cells from another individual (donor), commonly of the same species, has been transferred. Generally the MHC antigens, which may be Class I or Class II, will be matched, although one or more of the MHC antigens may be different in the donor as compared to the recipient. The graft recipient and donor are generally mammals, preferably human. Laboratory animals, such as rodents, e.g. mice, rats, etc. are of interest for drug screening, elucidation of developmental pathways, etc. For the purposes of the invention, the cells may be allogeneic, autologous, or xenogeneic with respect to the recipient.

### Methods of Identifying, Isolating and Enriching NSPCs

In one aspect, this application is directed to methods for identifying, isolating and enriching NSPCs from brain tissue samples. The methods comprise dissociating single cells from a brain tissue sample, contacting the single cells with a panel of antibodies thereby producing labeled single cells and selecting the cells based on the antibody labeling. The selected labeled single cells may then be used to generate neurospheres, for research, or may be used in the treatment of a neurological disorder or injury.

A variety of NSCPs may be isolated using the methods disclosed herein. Examples of NSCPs that can be isolated include, without limitation, bipotent glial progenitor (BP), ventricular radial glia (vRG), outer radial glia (ORG), astrocytes (AC), pre-oligodendrocyte precursor cells (pre-OPC), oligodendrocyte precursor cells (OPC), oligodendrocytes (OL), early excitatory neurons (early ExN), late excitatory neurons (late ExN), inhibitory neurons (InN), intermediate progenitor cells (IPC), etc.

NSPCs may be isolated from any suitable brain tissue sample using the methods disclosed herein. The brain tissue sample can be from any species including. without limitation, human, mouse, rat, non-human primate, etc. In some embodiments, the brain tissue sample is from a mouse. In some embodiments, the brain tissue sample is from a human.

When the brain tissue sample is from a human, the brain tissue sample may be from a specific time point in brain development. For instance, the human brain tissue sample may be from a fetal brain or an adult brain. In some embodiments, the brain tissue sample is from a fetal brain at the gestational age of 14-28 weeks. In some embodiments, the gestational age is from about 14-16 weeks, about 16-18 weeks, about 18-20 weeks, about 20-22 weeks, about 22-24 weeks, about 24-26 weeks or about 26-28 weeks. In some embodiments, the brain sample is from a fetal brain at the gestational age of less than 14 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks or greater than 28 weeks.

In order to isolate NSPC populations, single cells must be first dissociated from brain tissue samples. Single cells may be dissociated from brain tissue samples using mechanical and enzymatic dissociations. Methods of mechanical dissociation that find use in the present disclosure included, without limitation, chopping, dicing, mincing, crushing, etc. Following mechanical dissociation, brain tissue samples are further processed using enzymatic dissociation. In some embodiments, enzymatic dissociation comprises four steps. In the first step, the mechanically dissociated brain tissue sample is contacted with a first enzymatic solution comprising a salt solution, a thermolysin, and a DNase. In the second step, the cells from step 1 are removed from the first enzymatic solution and resuspended in a second enzymatic solution comprising a cell detachment solution, such as ACCUTASE™, and a DNase. In the third step, red blood cells are separated from the other single cells using centrifugation and a density gradient comprising a polysucrose solution such as Histopaque or Ficoll. In the fourth step, single cells are isolated from the buffy coat at the fluid interface within the density gradient.

Separation of the subject cell population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

The affinity reagents of particular interest are antibodies as affinity reagents. The details of the preparation of antibodies and their suitability for use as specific binding members are well known to those skilled in the art. Depending on the specific population of cells to be selected, one or more antibodies having specificity for the immunophenotypes disclosed below are contacted with the starting population of cells.

As is known in the art, the antibodies will be selected to have specificity for the relevant species, i.e. antibodies specific for human markers are used for selection of human cells; antibodies specific for mouse markers are used in the selection of mouse cells, and the like.

Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Non-limiting examples of useful fluorochromes include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, TurboYFP, mOrange, TurboRFP, tdTomato, TagRFP, dsRed2, mRFP, mCherry, mPlum mRaspberry, mScarlet, xanthene derivatives, cyanine derivatives, squaraine derivatives, naphthalene derivatives, coumarin derivatives, oxadiazole derivatives, anthracene derivatives, pyrene derivatives, oxazine derivatives, acridine derivatives, arylmethine derivative, tetrapyrrole derivatives, dipyrromethene derivatives, etc. Frequently each antibody is labeled with a different fluorochrome, to permit independent sorting for each marker.

The antibodies are added to a suspension of single cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbeccos Modified Eagle Medium (dMEM), Hank's Basic Salt Solution (HBSS), Dulbeccos phosphate buffered saline (dPBS), RPMI, Iscoves medium, PBS with 5 mM EDTA, etc., frequently supplemented with fetal calf serum, BSA, HSA, etc.

The antibodies added to the suspension of single cells may be in the form of an antibody panel. The antibody panel comprises one or more of a PROM1 (CD133), a CD24, a THY1 (CD90), a CXCR4, an EGFR, a PDGFRA, a CD45, a PECAM1 (CD31), a CD34, an ENG (CD105), or a GYPA (CD235a) antibody. In some embodiments, The antibody panel comprises a PROM1 (CD133), a CD24, a THY1 (CD90), a CXCR4, an EGFR, a PDGFRA, a CD45, a PECAM1 (CD31), a CD34, an ENG (CD105), and a GYPA (CD235a) antibody The contacting of the suspension of the single cells with the antibody panel produces labeled single cells.

Labeled single cells are then flow cytometrically selected based on their immunophenotype. In some embodiments, labeled single cells are selected using fluorescence activated cell sorting (FACS). In some embodiments, the NSCPs are selected based on positive selection markers. In some embodiments, the NSCPs are selected based on negative selection markers. In some embodiments, the NSCPs are selected based on a combination of positive and negative selection markers.

By a marker or combination of markers (e.g. an immunophenotype) it is meant that, in brain tissue samples comprising NSPCs, the marker or combination of markers are expressed selectively by the cells of the culture that will develop, are developing, and/or have developed into an NSPC from a specific population. It will be understood by those of skill in the art that the stated expression levels reflect detectable amounts of the marker protein on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs. yet still be characterized as “positive”.

Cells of interest, i.e. cells expressing the marker of choice, may be enriched for, that is, separated from the rest of the cell population, by a number of methods that are well known in the art. For example, flow cytometry, e.g. fluorescence activated cell sorting (FACS), may be used to separate the cell population based on the intrinsic fluorescence of the marker, or the binding of the marker to a specific fluorescent reagent, e.g. a fluorochrome-conjugated antibody, as well as other parameters such as cell size and light scatter. In other words, selection of the cells may be effected by flow cytometry. Although the absolute level of staining may differ with a particular fluorochrome and reagent preparation, the data can be normalized to a control. To normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unlabeled cells. When displayed in this manner, it is clear that the cells falling in the highest log of staining intensity are high, while those in the lowest intensity are negative. The “low” positively stained cells have a level of staining above the brightness of an isotype matched control, but are not as intense as the most brightly staining cells normally found in the population. An alternative control may utilize a substrate having a defined density of marker on its surface. for example a fabricated bead or cell line, which provides the positive control for intensity.

Other methods of separation, i.e. methods by which selection of cells may be effected, based upon markers include, without limitation, magnetic activated cell sorting (MACS), immunopanning, and laser capture microdissection.

The immunophenotype of the NSPC define which population of NSPC the cells belong to. Ventricular radial glia may be defined by the immunophenotype CD24^{-/lo}THY1^{-/lo} EGFRhi. Outer radial glia may be defined by the immunophenotype CD24^{-/lo} THY1^{-/lo} EGFR−. Astrocytes may be defined by the immunophenotype CD24^{-/lo} THY1^{-/lo} EGFR+ CXCR4+. Pre-oligodendrocyte precursor cells may be defined by the immunophenotype THY1hi EGFR+ PDGFRA+. Oligodendrocyte precursor cells may be defined by the immunophenotype THY1 hi EGFR− PDGFRA+. Oligodendrocytes may be defined by the immunophenotype THY1hi EGFR− PDGFRA−. Early excitatory neurons may be defined by the immunophenotype CD24+ THY1^{-/lo} CXCR4− EGFR−. Late excitatory neurons may be defined by the immunophenotype CD24+ THY1^{-/lo} CXCR4− EGFR+. Inhibitory neurons may be defined by the immunophenotype CD24+ THY1^{-/lo} CXCR4+ EGFR−. Bipotent glial progenitors may be defined by the immunophenotype THY1^{hi}EGFR^{hi}PDGFRA⁻.

In some embodiments, ventricular radial glia are further defined by expression of transcripts of one or more of SOX2, GFAP, VIM, PAX6, CRYAB or FBXO32. In some embodiments, outer radial glia are further defined by expression of transcripts of one or more of SOX2, GFAP, VIM, HOPX, TNC, or LIFR. In some embodiments, astrocytes are further defined by expression of transcripts of one or more of SOX2, GFAP, VIM. PAX3, NTRK2, ZIC1, CXCR4, or EDNRB. In some embodiments, pre-OPCs are further defined by expression of transcripts of one or more of OLIG1, OLIG2, SOX10, EGFR, MKI67, or PCNA. In some embodiments, OPCs are further defined by expression of transcripts of one or more of OLIG1, OLIG2, SOX10, PDGFRA, or PCDH15. In some embodiments, OLs are further defined by expression of transcripts of one or more of OLIG1, OLIG2, SOX10, MYRF, or MBP. In some embodiments, early ExNs are further defined by expression of transcripts of one or more of DCX, SOX4, SOX11, SSTR2, or NEUROD2. In some embodiments, late ExNs are further defined by expression of transcripts of one or more of DCX, SOX4, SOX11, TBR1, SYT4, or SATB2. In some embodiments, InNs are further defined by expression of transcripts of one or more of DCX, SOX4, ERBB4, or SOX11. In some embodiments, the transcripts of NSCPs are measured using single cell RNA sequencing (scRNA-seq).

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscoves medium, etc., frequently supplemented with fetal calf serum. In some embodiments, the separated cells may be maintained in the form of neurospheres. Methods of neurosphere generation are well known in such as those disclosed in disclosed in Weiss et al., U.S. Pat. No. 5,750,376 and Weiss et al., U.S. Pat. No. 5,851,832, Marshal et al. (Methods Mol Biol. 2008; 438:135-150), each of which is specifically incorporated by reference herein.

NSCPs populations isolated using the methods disclosed herein are of high purity. For instance, the NSPC populations may have a purity of 75%, 80%, 85%, 90%, 95%, or a purity of greater than 95%.

In some embodiments. NSCPs isolated using the above mentioned methods have a greater likelihood to produce neurospheres relative to NSCPs isolated using different methods. For instance, NSCPs isolated using the above mentioned methods may have a greater than about 1.5, 2, 3, 4, 5, 6, 7, 8, 9, 10 or greater than 10 times higher likelihood of forming neurospheres relative to NSPCs isolated using a different method. In some embodiments, among the isolated NSCPs using the methods disclosed herein, NSCPs comprising an immunophenotype comprising either EGFRhi or EGFR+have a higher likelihood of producing neurospheres. For example, isolated NSPCs comprising an immunophenotype comprising either EGFR^{hi} or EGFR⁺ have a greater than about 1.5, 2, 3, 4, 5, 6, 7, 8, 9, 10 or greater than 10 times higher likelihood of forming neurospheres relative to NSPCs isolated that do not comprise an immunophenotype comprising either EGFR^{hi} or EGFR⁻.

### Methods of Treating a Neurological Disorder

NSPC populations described herein may be used in cell replacement or cell transplantation therapy to treat diseases, particularly neurological diseases or disorders of the CNS. In some embodiments, the cell transplantation therapy is a neuron transplantation therapy. In some embodiments the cell transplantation therapy is a glial cell transplantation therapy.

Subjects in need of neuron transplantation therapy, e.g. a subject suffering from a neurological condition associated with the loss of neurons. glial cells, astrocytes, oligodendrocytes, etc. or with aberrantly functioning cells, could especially benefit from therapies that utilize cells derived by the methods of the invention. Examples of such diseases, disorders and conditions include neurodegenerative diseases (e.g. Parkinson's Disease, Alzheimer's Disease, Huntington's Disease, Amyotrophic Lateral Sclerosis (ALS), Spielmeyer-Vogt-Sjögren-Batten disease (Batten Disease), Frontotemporal Dementia with Parkinsonism, Progressive Supranuclear Palsy, Pick Disease, prion diseases (e.g. Creutzfeldt-Jakob disease), Amyloidosis, glaucoma, diabetic retinopathy, age related macular degeneration (AMD), and the like); neuropsychiatric disorders (e.g. anxiety disorders (e.g. obsessive compulsive disorder), mood disorders (e.g. depression), childhood disorders (e.g. attention deficit disorder, autistic disorders), cognitive disorders (e.g. delirium, dementia), schizophrenia, substance related disorders (e.g. addiction), eating disorders, and the like); channelopathies (e.g. epilepsy, migraine, and the like); lysosomal storage disorders (e.g. Tay-Sachs disease, Gaucher disease, Fabry disease, Pompe disease, Niemann-Pick disease, Mucopolysaccharidosis (MPS) & related diseases, and the like); autoimmune diseases of the CNS (e.g. Multiple Sclerosis, encephalomyelitis, paraneoplastic syndromes (e.g. cerebellar degeneration), autoimmune inner ear disease, opsoclonus myoclonus syndrome, and the like); cerebral infarction, stroke, and spinal cord injury.

In some approaches, the NSPCs may be transplanted directly to an injured site to treat a neurological condition, see, e.g., Morizane et al., (2008), Cell Tissue Res., 331(1):323-326; Coutts and Keirstead (2008), Exp. Neurol., 209 (2): 368-377; Goswami and Rao (2007), Drugs, 10(10):713-719. For example, for the treatment of Parkinson's disease, neurons may be transplanted directly into the striate body of a subject with Parkinson's disease. As another example, for treatment of ALS, corticospinal motor neurons may be transplanted directly into the motor cortex of the subject with ALS. In other approaches, the cells derived by the methods of the invention are engineered to respond to cues that can target their migration into lesions for brain and spinal cord repair; see, e.g., Chen et al. (2007) Stem Cell Rev. 3(4):280-288.

The NSPCs may be administered in any physiologically acceptable medium. They may be provided prior to differentiation, i.e. they may be provided in an undifferentiated state and allowed to differentiate in vivo, or they may be allowed to differentiate for a period of time ex vivo and provided following differentiation. They may be provided alone or with a suitable substrate or matrix, e.g. to support their growth and/or organization in the tissue to which they are being transplanted. Usually, at least 1×10⁵ cells will be administered, preferably 1×10⁶ or more. The cells may be introduced to the subject via any of the following routes: parenteral, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5385582, incorporated herein by reference); by bolus injection, e.g. by a syringe, e.g. intravitreally or intracranially; by continuous infusion, e.g. by cannulation, e.g. with convection (see e.g. U.S. application Ser. No. 20/070,254842, incorporated here by reference); or by implanting a device upon which the cells have been reversibly affixed (see e.g. U.S. application Ser. Nos. 20/080,081064 and 20090196903, incorporated herein by reference).

For some central nervous system conditions involving pathogenic expansion of cell types at a particular stage of stem or progenitor cell type, and the cell surface molecules of these cells can be targets for immune therapy; these can include antibodies or T cells or checkpoint inhibitors of macrophages, etc.

The number of administrations of treatment to a subject may vary. Introducing the NSPCs into the subject may be a one-time event; but in certain situations, such treatment may elicit improvement for a limited period of time and require an on-going series of repeated treatments. In other situations, multiple administrations of the NSPCs may be required before an effect is observed. The exact protocols depend upon the disease or condition, the stage of the disease and parameters of the individual subject being treated.

For some central nervous system conditions, it may be necessary to formulate the composition comprising the NSCPs isolated using the methods disclosed herein to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. A BBB disrupting agent can be co-administered with the therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of the composition behind the BBB may be by local delivery, for example by intrathecal delivery, e.g. through an Ommaya reservoir (see e.g. U.S. Pat. Nos. 5,222,982 and 5385582, incorporated herein by reference); by bolus injection, e.g. by a syringe, e.g. intravitreally or intracranially; by continuous infusion, e.g. by cannulation. e.g. with convection (see e.g. US Application No. 20070254842, incorporated here by reference); or by implanting a device upon which the NR pharmaceutical composition has been reversibly affixed (see e.g. US Application Nos. 20080081064 and 20090196903, incorporated herein by reference).

The calculation of the effective amount or effective dose of the composition comprising the NSCPs isolated using the methods disclosed herein to be administered is within the skill of one of ordinary skill in the art, and will be routine to those persons skilled in the art. Needless to say, the final amount to be administered will be dependent upon the route of administration and upon the nature of the disorder or condition that is to be treated.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

The features and advantages of the present invention will be more clearly understood by reference to the following examples, which are not to be construed as limiting the invention.

### EXPERIMENTAL

### Example 1

The human brain undergoes rapid development at midgestation from a pool of neural stem and progenitor cells (NSPCs), which give rise to the neurons, oligodendrocytes, and astrocytes of the mature brain. However, not all of the intermediate steps between neural stem cells (NSCs) and mature brain cells are known, and functional study of these cell types has been hampered by a lack of precise purification methods. We describe a method for prospectively isolating nine distinct NSPC types from the developing human brain using combinations of cell surface markers. Among the neural cells, CD24⁻THY1^{-/lo} cells were enriched for radial glia, which robustly engrafted, migrated, and differentiated into all three neural lineages in the mouse brain. THY1^{hi} cells marked unipotent oligodendrocyte precursors committed to an oligodendroglial fate, and CD24⁺THY1^{-/lo} cells marked committed excitatory and inhibitory neuronal lineages. Notably, we identify and functionally characterize a transcriptomically-distinct THY1^{hi}EGFR^{hi}iPDGFRA⁻ bipotent glial progenitor, which is lineage-restricted to astrocytes and oligodendrocytes, but not neurons. Our study provides a framework for the functional study of distinct cell types in human neurodevelopment.

The prenatal human brain undergoes rapid development during the second trimester from a pool of neural stem cells (NSCs) and downstream progenitor cells which give rise to the neurons, oligodendrocytes, and astrocytes of the mature brain. Functional study of these cell types has been hampered by a lack of precise purification methods. We describe a fluorescence-activated cell sorting (FACS) based method for the prospective isolation of nine distinct neural stem and progenitor cell (NSPC) populations from the developing human cortex using combinations of cell-surface markers that constitute their immunophenotype. Broadly, these cell types include a CD24⁻THY1^{-/lo} compartment enriched in ventricular and outer radial glia (vRG, oRG) as well as astrocytes, a THY1^{hi} compartment encompassing all oligodendrocyte lineages, and a CD24⁺THY1^{-/lo} compartment enriched in excitatory and inhibitory neuronal lineages. The purity of isolated NSPCs was assessed by correlating the expressed transcriptomes of individual index-sorted cells with their immunophenotype. We confirmed the functional purity of isolated NSPCs regarding their lineage output potential through in vitro differentiation assays, and through in vivo transplantation into the lateral ventricles of neonatal immunodeficient mouse brains. Transplanted radial glia subsets robustly engrafted, migrated, and differentiated in a site appropriate manner into all three neural lineages in the mouse brain 6 months post-engraftment. In contrast, transplanted oligodendrocyte precursors were unipotent, exclusively giving rise to oligodendrocytes. Notably, we identify, prospectively isolate, and functionally characterize a transcriptomically-distinct bipotent THY1^{hi}EGFR^{hi}PDGFRA⁻ glial progenitor (GP), which are lineage-restricted to astrocytes and oligodendrocytes but not neurons. The methodology within this study thus provides a framework for the functional study of distinct cell types in human neurodevelopment.

Here, we combine high-dimensional flow cytometry with single cell transcriptomics to comprehensively purify and functionally characterize neural stem cells and distinct downstream progenitor subsets from the developing human brain during mid-gestation (GW16-18). We utilize index-sorting based on the combinatorial expression of cell-surface markers (i.e. immunophenotype) to directly link surface marker expression to the expressed transcriptomes of single cells, allowing for rigorous validation of sort purity. Our tissue processing and sorting strategy allows for the simultaneous purification of nine defined NSPC populations, including radial glia, neuron precursors, oligodendrocyte precursors, and astrocyte lineage cells, reflective of a NSC lineage hierarchy as described in other tissue stem cell systems. We further demonstrate their functional properties in vitro, and in vivo. Within this framework, we also identify a novel bipotent glial progenitor that gives rise exclusively to astrocytes and oligodendrocytes. Our findings aim to lay the groundwork for isolating NSPCs to establish the self-renewal and differentiation potential of these cells, and for future molecular and cellular studies on human neurodevelopment and the development of cell transplantation-based therapeutic regimens.

### RESULTS

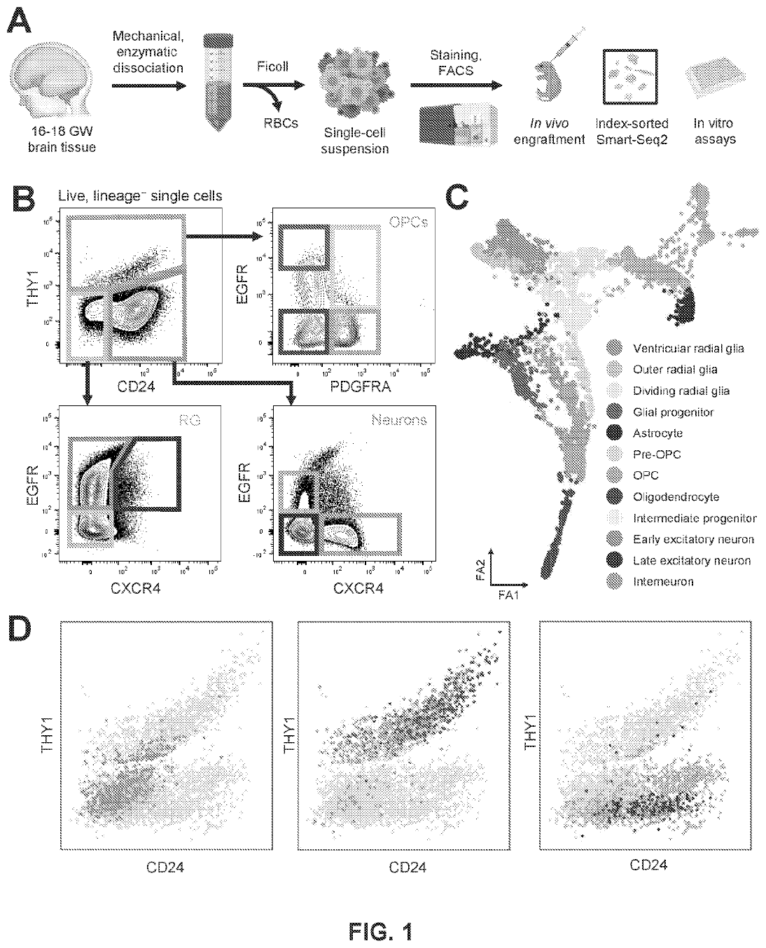

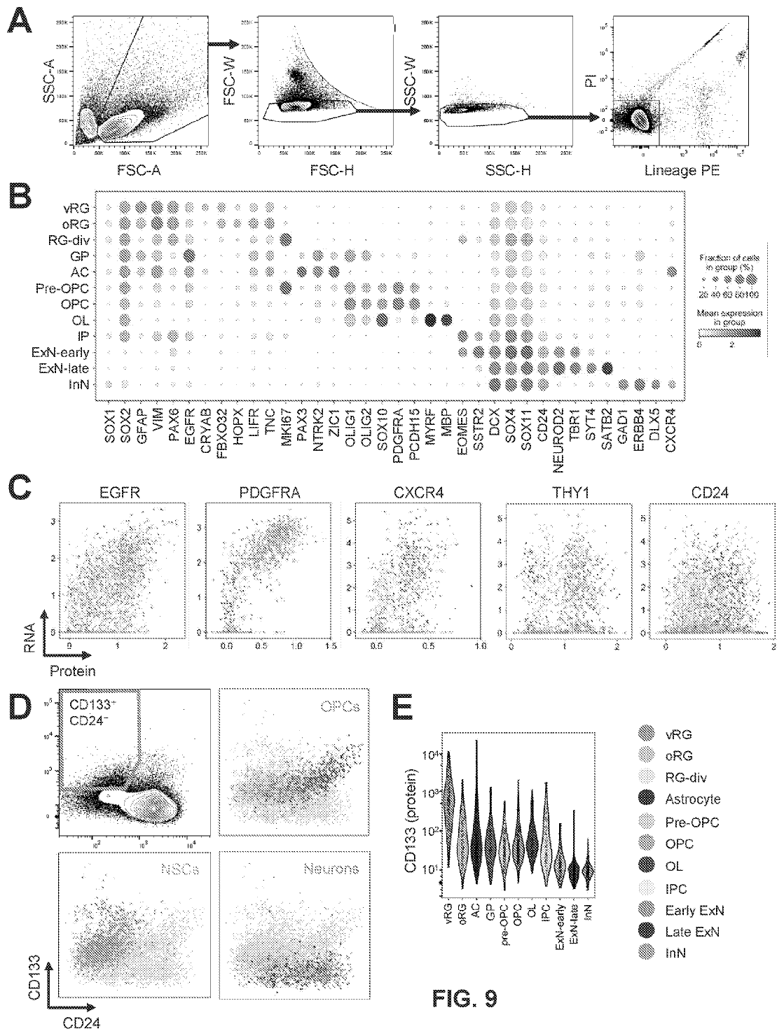

Immunophenotypic definition of neural stem and progenitor cell types. To purify cell-intrinsically distinct putative NSPC fractions, we mechanically and enzymatically dissociated GW16-18 human brain tissue into a single-cell suspension (FIG. 1A). The cortex was dissected out prior to dissociation whenever possible. The cells were stained with a panel consisting of antibodies against PROM1 (CD133), CD24, THY1 (CD90), CXCR4, EGFR, PDGFRA, and non-neural lineages (CD45, PECAM1 [CD31], CD34, ENG[CD105], and GYPA [CD235a]). The stained cell suspension was analyzed using fluorescence-activated cell sorting (FACS) (FIG. 1B, FIG. 9A). The observed immunophenotypic profile was extremely consistent across over a dozen samples of this gestational age group. Various populations that stratified based on cell-surface marker expression (immunophenotype) were index-sorted into 96-well plates for full-length single-cell RNA-sequencing (scRNA-seq) using Smart-seq2 or Smart-seq3. Index-sorting records the fluorescence intensities for each sorted single cell, allowing for direct mapping between surface marker profile and the expressed transcriptome.

Unsupervised clustering of single-cell transcriptomes identified various putative NSPC populations, which we annotated using input from a combination of functional assays and expressed mRNAs of known marker genes (FIG. 1C; FIG. 9B). These transcriptomically-defined cell types include putative ventricular radial glia (vRG), outer radial glia (oRG), astrocytes (AC), pre-oligodendrocyte precursor cells (pre-OPC), OPCs, oligodendrocytes (OLs), intermediate progenitor cells (IPCs), and excitatory and inhibitory neuronal lineage cells (ExN, InN) (see details below).

Index-sort data allowed for each individual sequenced cell to be mapped back to its original immunophenotype. RNA and cell-surface protein expression did not always correlate, most notably with the glycosylphosphatidylinositol (GPI)-anchored surface molecules THY1 and CD24 (FIG. 9C). Using the index-sort information, we found that the putative NSCs were CD24⁻THY1^{-/lo}, the putative OPCs were THY1^{hi}, and the putative neuronal lineage cells were CD24⁺THY1^{-/lo} (FIG. 1D), with additional heterogeneity present in each compartment. We have previously reported that CD133⁺CD24⁻ cells are highly enriched for NSCs; indeed, our index-sort analysis confirms that this gate largely consists of vRG (FIG. 9D, E). Our gating strategy resulted in remarkably pure populations of sorted cells as validated by scRNA-seq, allowing for prospective isolation of various putative NSPCs for downstream functional assays.

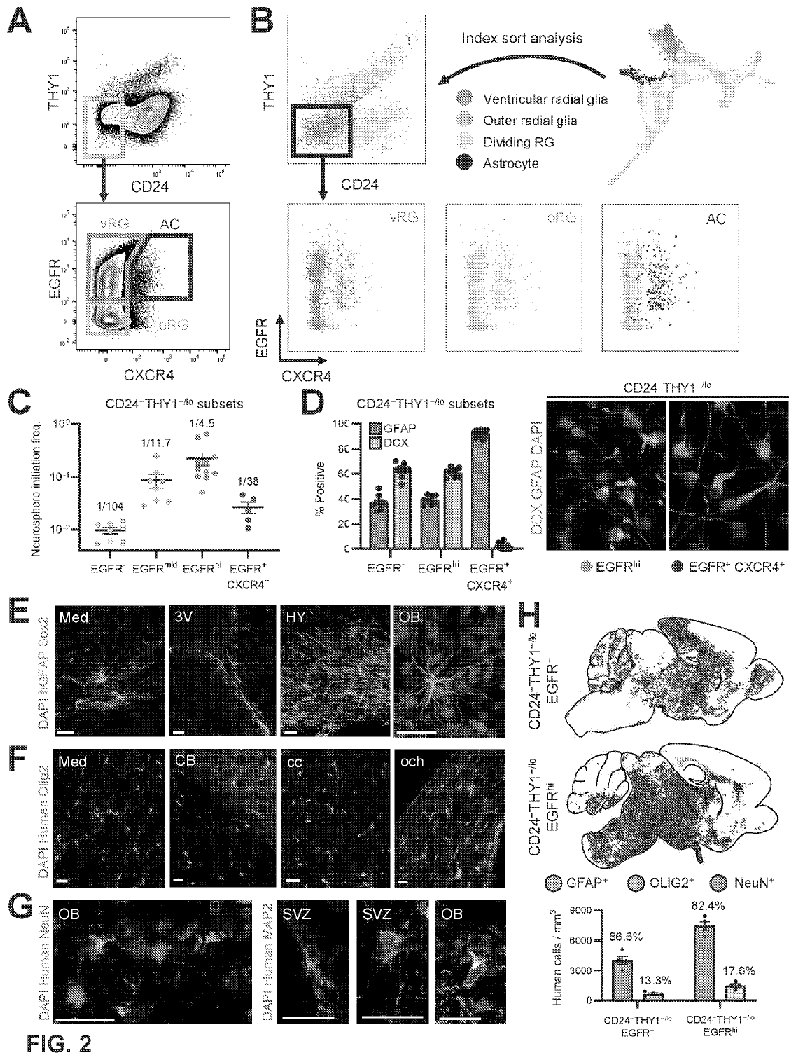

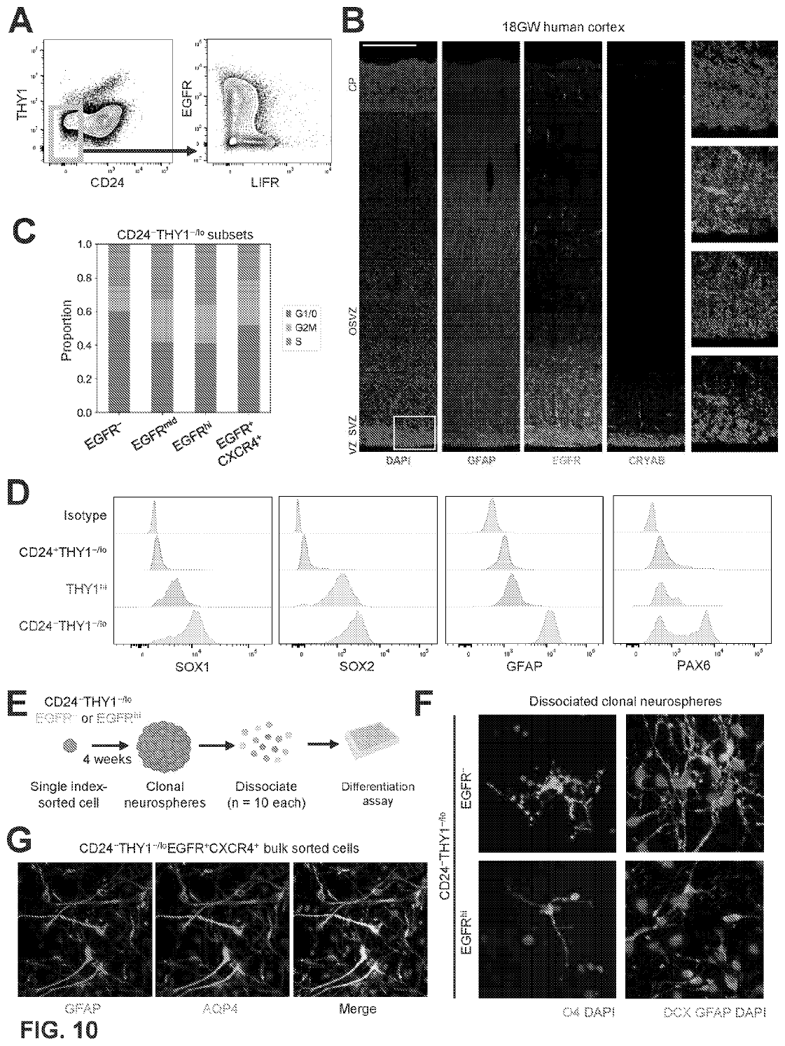

CD24⁻THY1^{-/lo} expression identifies phenotypic NSCs. Index-sort data showed that putative NSCs, broadly expressing SOX2, GFAP, and VIM transcripts, were enriched by the CD24⁻THY1^{-/lo} gate, with further immunophenotypic heterogeneity observed in cell-surface expression of EGFR and CXCR4 (FIG. 2A). Within the CD24⁻THY1^{-/lo} gate, the EGFR^{hi} population was enriched for putative vRG (expressing CRYAB and FBXO32 transcripts), while the EGFR⁻ population was enriched for putative oRG (expressing HOPX and LIFR transcripts FIG. 2B, and LIFR protein, FIG. 10A). Histological analysis confirmed that EGFR is expressed in the ventricular and subventricular zones (VZ/SVZ) but absent in the outer subventricular zone (OSVZ) (FIG. 10B). In the VZ/SVZ, EGFR colocalized with GFAP and the vRG-specific marker CRYAB, supporting our finding that EGFR distinguishes vRG from ORG. We also observed a population of EGFR⁺CXCR4⁺ putative astrocyte (AC) lineage enriched for PAX3 and EDNRB transcripts. Intracellular staining followed by flow cytometry confirmed that, similar to their expression at the transcript level (FIG. 9B), the CD24⁻THY1^{-/lo} cells also highly expressed the protein versions of known NSC marker genes, including SOX1, SOX2, GFAP, and PAX6 (FIG. 10D). Notably, the expression of PAX3 transcript was restricted to the putative AC lineage (FIG. 9B), suggesting that PAX3 may have a role in early lineage bifurcation between the NSC and AC lineages. This finding is in contrast with studies in mouse showing that Pax3 downregulation is associated with differentiation towards an astrocytic fate. It is possible that our AC population represents an early progenitor population where PAX3 induction is initially required before being downregulated with maturation. Alternatively, there could be considerable cellular heterogeneity within the AC lineage with respect to PAX3 expression in human prenatal brains.

To assess whether our putative NSC subsets are functional, we next assessed their self-renewal and differentiation capabilities in vitro and in vivo. We have previously shown that CD133⁺CD24⁻ cells can self-renew and be maintained in the presence of LIF, FGF, and EGFR and expanded as neurospheres. Consistent with our previous results, CD24⁻THY1^{-/lo} putative NSCs readily formed neurospheres when cultured. In addition, in vitro limiting dilution assays showed that within the CD24⁻THY1^{-/lo} gate, the EGFR^{hi} population possesses markedly higher neurosphere initiation frequency (1 in 4.5) compared to the EGFR⁻ population (1 in 10⁴) (FIG. 2C)—a difference that was not attributable to cell cycle (FIG. 10C). To assess the functional behavior of individual CD24⁻THY1^{-/lo}putative NSCs in vitro, we generated clonal neurospheres from single sorted cells. We dissociated individual single-cell-derived clonal neurospheres and subjected them to differentiation conditions. Both EGFR⁻ and EGFR^{hi} NSC clones could be differentiated into DCX⁺, GFAP⁺, and rare O4⁺ cells, demonstrating their competence to give rise to neuron, astrocyte, and oligodendrocyte lineages (FIG. 10E, F). In contrast, bulk-sorted EGFR⁺CXCR4⁺ cells gave rise almost exclusively to GFAP⁺ cells which also stained positive for AQP4, suggesting they are largely committed to an astrocytic fate (FIG. 2D, FIG. 10G).

To functionally characterize our putative NSCs in vivo, we transplanted acutely isolated cells directly, without intervening culture, into the lateral ventricles of neonatal NOD-scid-IL2Rg^{null} (NSG) immunodeficient mice. After 6 months, we assessed the level of engraftment by immunofluorescent (IF) staining for human-specific antigens. Transplanted NSCs migrated and engrafted extensively throughout the brain and differentiated to give rise to all three major neural lineages: GFAP⁺ astrocytes/NSCs, OLIG2⁺ oligodendrocytes, and NeuN⁺ or MAP2⁺ neurons (FIG. 2f-h). Remarkably, the CD24⁻THY1^{-/lo} EGFR^{hi} NSCs resulted in significantly higher levels of engraftment compared to CD24⁻THY1^{-/lo} EGFR⁻ NSCs, with 1.8-fold more OLIG2⁺ cells and 2.3-fold more GFAP⁺ cells observed across four replicates. This result is consistent with previous studies in the mouse demonstrating the role of EGFR in promoting proliferation and astrogliogenesis. Collectively, our in vitro and in vivo results confirm that our putative NSC fractions are indeed functional with respect to their self-renewal capability and differentiation potential. In addition, among the CD24⁻THY1^{-/lo} NSCs, EGFR^{hi} cells are likely enriched for relatively primitive multipotent NSCs compared to EGFR⁻ cells.

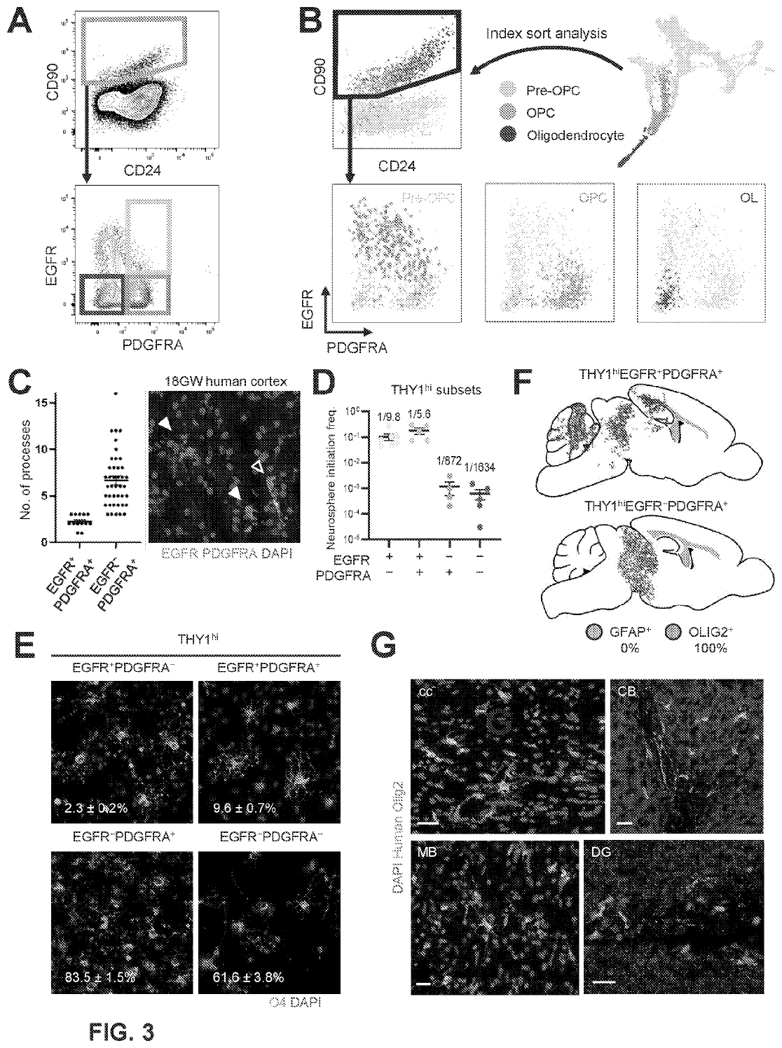

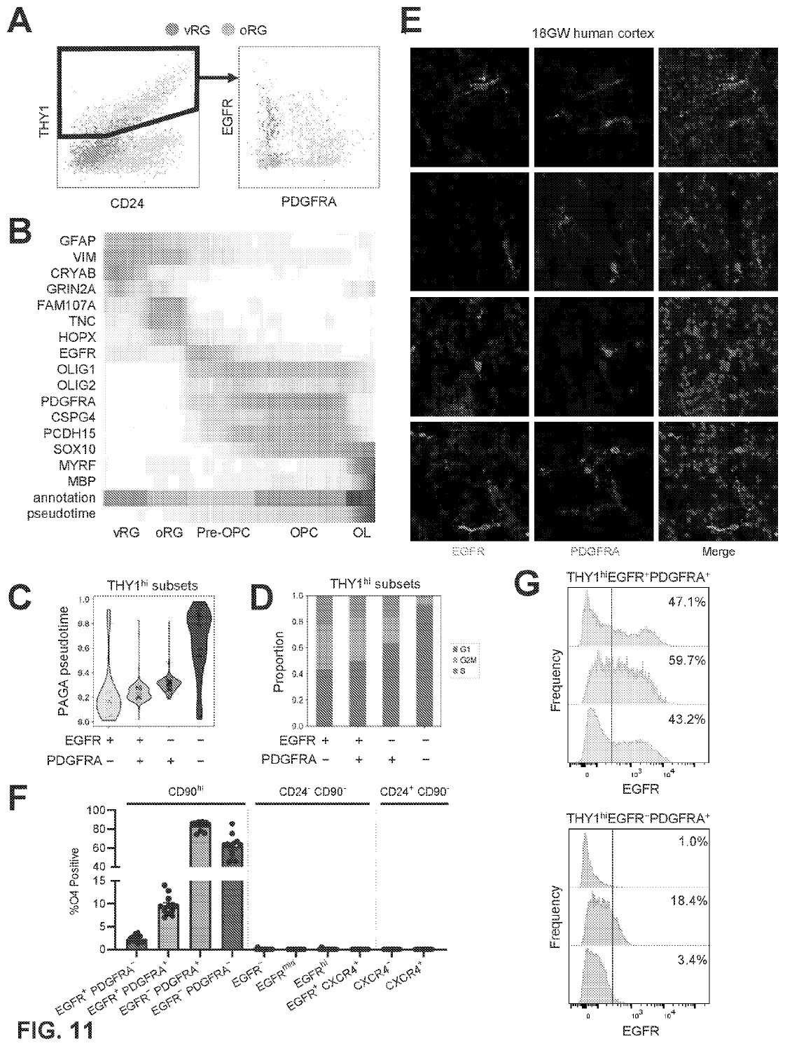

THY1^{hi} expression identifies oligodendrocyte lineages. Index-sort data combined with unsupervised clustering showed that the putative oligodendrocyte (OL) lineages, broadly expressing OLIG1, OLIG2, and SOX10 transcripts, was enriched by the THY1^{hi} gate. This gate highly enriched all OL lineages, suggesting THY1 to be a pan-oligodendrocyte lineage marker at least at this developmental stage. Among the THY1hi cells, we observed additional heterogeneity with respect to cell-surface expression of EGFR and PDGFRA, resulting in four immunophenotypic populations: THY1^{hi}EGFR⁺PDGFRA⁻ (T^{hi}E⁺P⁻), THY1^{hi}EGFR⁺PDGFRA⁺ (T^{hi}E⁺P⁺), THY1^{hi}EGFR⁻PDGFRA⁺ (T^{hi}E⁻P⁺), and THY1^{hi}EGFR⁻PDGFRA⁻ (T^{hi}E⁻P⁻) (FIG. 3A). Index-sort analysis showed that the T^{hi}E⁻P⁺ gate highly enriched for putative pre-OPCs (expressing EGFR, PDGFRA, and PCNA transcripts), the T^{hi}E⁻P⁺ gate for putative OPCs (expressing PDGFRA and PCDH15 transcripts), and the T^{hi}E⁻P⁻ gate for more mature oligodendrocytes (expressing MYRF and MBP transcripts) (FIG. 3B). The T^{hi}E⁺P⁻ gate consisted of a heterogeneous population likely consisting of both putative OPCs and NSCs (FIG. 3B, FIG. 11A), further discussed below. Consistent with our gene expression-based inference, pseudotime analysis also recapitulated the oligodendrocyte maturation trajectory (FIG. 11B). Plotting the pseudotime by immunophenotype shows a gradient of maturation from the T^{hi}E⁺P⁺ to the T^{hi}E⁻P⁺, and then to the T^{hi}E⁻P⁻gates (FIG. 11C). Indeed, the T^{hi}E⁺P⁺ gate was enriched for actively-cycling cells (FIG. 11D), suggesting that they are a transit-amplifying population that expands the OPC pool. likely corresponding to pre-OPCs. Histological analysis confirmed the presence of both EGFR⁻PDGFRA⁺ and EGFR⁺PDGFRA⁺ cells with OPC-like morphology in the fetal human cortex (FIG. 3C, FIG. 11E). EGFR+PDGFRA⁺ cells had simpler morphology compared to EGFR⁻PDGFRA⁺ cells (average 2.2 vs. 6.7 processes per cell, respectively), suggesting that the former represent a less mature cell type.

We next assessed the functional behavior of the putative OPC subsets in vitro. Limiting dilution assays showed that the Th E⁺P⁻ and the T^{hi}E⁺P⁺ cells had a high neurosphere initiation rate (1 in 9.8 and 1 in 5.6, respectively), in contrast to the T^{hi}E⁻P⁻ and the T^{hi}E⁻P⁻ cells (1 in 872 and 1 in 1634, respectively) (FIG. 3D), demonstrating that EGFR expression marks a more proliferative pre-OPC subtype. Next, we sorted each of the four OPC populations and cultured them in the absence of growth factors to induce differentiation. Subsequent IF imaging of resulting cells post-differentiation revealed O4⁺ cells with distinctive oligodendroglial morphology (FIG. 3E). When the T^{hi}E⁺P⁻, T^{hi}E⁺P⁺, and T^{hi}E⁻P⁺ populations were subjected to the in vitro differentiation, the resulting proportions of O4⁺ cells showed an increasing trend from 2.3% to 9.6%, and then to 83.5%, respectively. The T^{hi}E⁻P⁻ cells did not survive well in culture likely due to their more mature state-nevertheless, the survivors still gave rise to 61.6% O4⁺ cells in our culture conditions (FIG. 3E). Strikingly, no other populations gave rise to any appreciable numbers of O4⁺ cells (FIG. 11F). To determine whether the putative pre-OPCs and OPCs are lineally related or represent interconvertible states, we reanalyzed sorted T^{hi}E⁺P⁺ and T^{hi}E⁻P⁺ populations after 5 days of in vitro differentiation using flow cytometry. In samples from three separate donors, while the T^{hi}E⁺P⁺ putative pre-OPCs gave rise to both EGFR⁺ and EGFR⁻ cells, the T^{hi}E⁻P⁺ putative OPCs remained EGFR⁻, suggesting a hierarchical organization (FIG. 11G). These results provide supportive evidence for the maturation trajectory predicted by the pseudotime analysis described above.

To assess their functional behavior in vivo, we then transplanted the THY1hi OPC subsets into the lateral ventricles of neonatal NSG mice. The T^{hi}E⁺P⁺ and T^{hi}E⁻P⁺ subsets engrafted robustly in the brain (FIG. 3F, G), and remarkably, gave rise exclusively to OLIG2⁺ cells, with no observed GFAP⁺ or NeuN/MAP2⁺ cells, suggesting that these cells are indeed committed to an oligodendroglial fate. The T^{hi}E⁻P⁻ cells did not engraft, possibly because they are too mature and post-mitotic. In summary, our collective in vitro and in vivo results confirm that the purified OPC subsets are engraftable and are indeed lineage committed to form only oligodendrocytes.

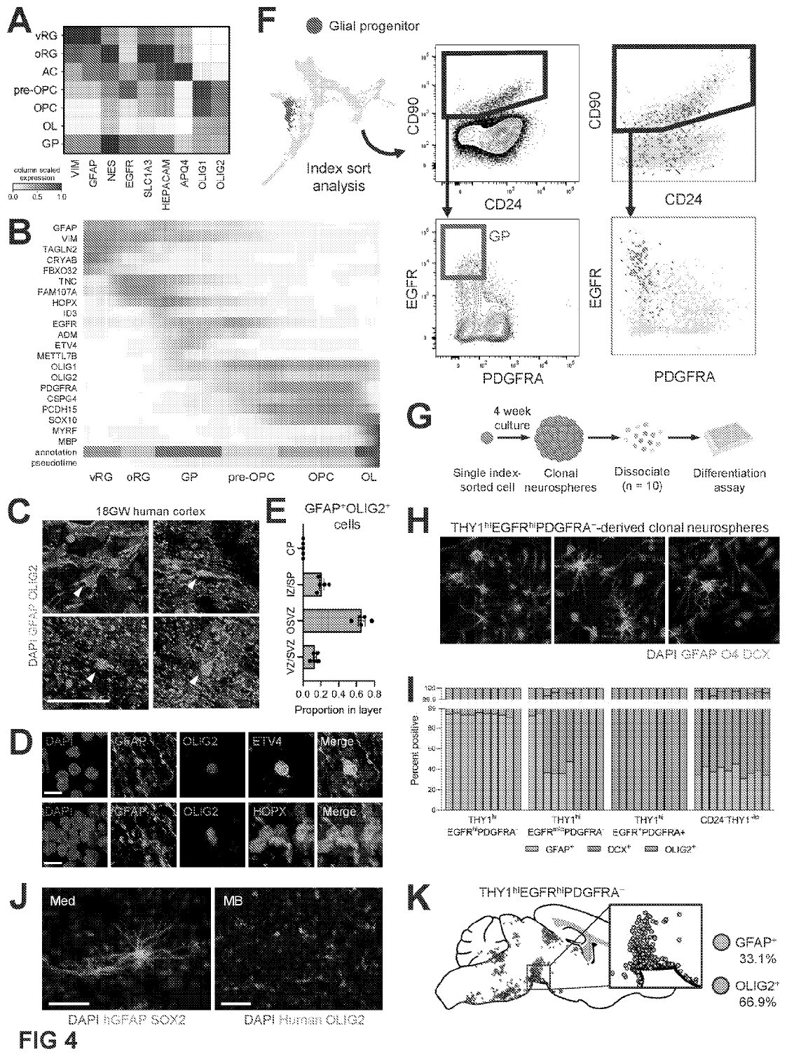

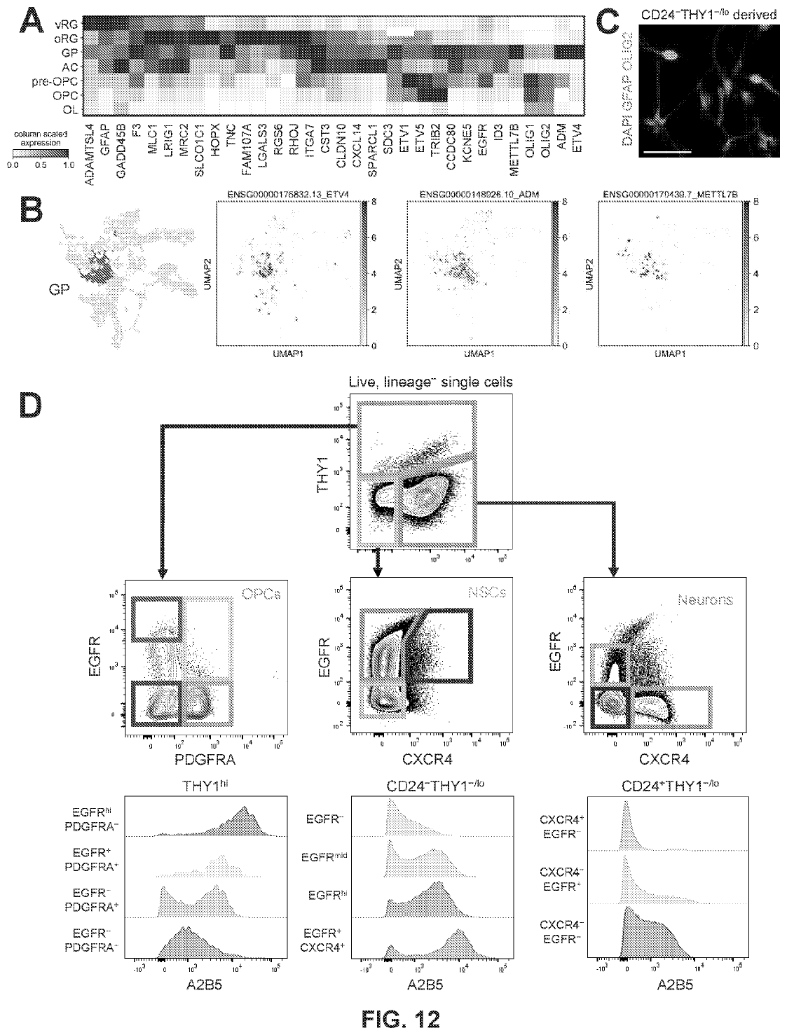

Identification of a bipotent glial progenitor. Within our scRNA-seq data, we identified a cluster of cells that expressed transcripts for genes characteristic of both astrocyte (GFAP, SLC1A3, HEPACAM, AQP4) as well as oligodendrocyte (OLIG1, OLIG2) lineages (FIG. 4A, FIG. 12A), leading us to hypothesize it as a putative bipotent glial progenitor (GP). Pseudotime trajectory analysis showed that these putative GPs maintain some oRG markers (HOPX, TNC) but also turn on other unique markers (ETV4, ADM, METTL7B) not found in other neural cell types (FIG. 4B, FIG. 12B). Through confocal imaging of fetal brain tissue, we were able to identify cells that stained positive for both GFAP and OLIG2 in the cortex (FIG. 4C). Further immunostaining confirmed that GFAP⁺OLIG2⁺ cells also stain positive for HOPX and ETV4 (FIG. 4D), consistent with our transcriptomic findings. Unlike HOPX, which is also expressed in ORG, ETV4 staining was specific to GFAP⁺OLIG2⁺ cells, validating it as a cell type-specific transcription factor for the putative GPs. These GFAP⁺OLIG2⁺ cells were preferentially located in the OSVZ, with lesser numbers found in the VZ/SVZ and the subplate, and none found in the cortical plate (FIG. 4E). This anatomical bias as well as their residual expression of ORG markers suggest that GPs may preferentially arise from oRG, though it does not exclude the possibility they may also arise directly from vRG. Indeed, in vitro differentiation of CD24^{-l THY}1^{-/lo} radial glia gives rise to some GFAP⁺OLIG2⁺ cells (FIG. 12C), supporting their hierarchical relationship.

Even with this transcriptomic and histologic data, functional characterization of our putative GP would only be possible if they can be prospectively isolated for downstream experiments. Fortunately, index-sort analysis showed that this cell type was enriched within the THY1^{hi}EGFR^{hi}PDGFRA⁻ gate (FIG. 4E), thus allowing for their prospective isolation. To assess the developmental potential of this cell type, we index-sorted single THY1^{hi}EGFR^{hi}PDGFRA⁻ cells and grew them into clonal neurospheres. We dissociated the clonal neurospheres and subjected the cells to differentiation in vitro by cytokine withdrawal (FIG. 4F). Strikingly, nine out of ten clonal neurospheres derived from THY1^{hi}EGFR^{hi}PDGFRA⁻ cells gave rise to both OLIG2⁺ oligodendroglial and GFAP⁺ astrocytic cells, but no DCX⁺ neuronal cells after differentiation (FIG. 4G, H), suggesting that at a clonal level a significant majority of the THY1^{hi}EGFR^{hi}PDGFRA⁻ cells are restricted to oligodendroglial and astrocytic fates. One of the ten neurospheres gave rise exclusively to OLIG2⁺ cells, indicating some unipotent pre-OPCs co-purified within this gate (FIG. 3B). In contrast, clonal neurospheres derived from THY1^{hi}EGFR⁺PDGFRA⁺ (T^{hi}E⁺P⁻) cells—which we have identified above as pre-OPCs and OPCs—gave rise exclusively to OLIG2⁺cells. Clonal neurospheres derived from the CD24⁻THY1^{-/lo} population enriched in radial glia gave rise to all three neural lineages, including DCX⁺, GFAP⁺, and rare OLIG2⁺ cells (FIG. 4H, FIG. 10E). Clonal neurospheres derived from THY1^{hi}EGFR^{mid}PDGFRA⁻ cells gave rise to a mix of unipotent, bipotent, and tripotent colonies, consistent with the index sort analysis showing that this gate consists of heterogeneous mix of pre-OPCs, glial progenitors, and radial glia (FIG. 3B, FIG. 11A, FIG. 4C).

To assess the in vivo behavior of the putative glial progenitors, we transplanted acutely-isolated THY1^{hi}EGFR^{hi}PDGFRA⁻ cells into the lateral ventricles of neonatal NSG mice. Six months post-transplant, we observed engraftment of both GFAP⁺ and OLIG2⁺ human cells (FIG. 4I, J). None of the engrafted human cells stained positive for either DCX or MAP2. Though it is not technically feasible to transplant single human cells, these transplant assays are still consistent with the identity of a bipotent glial progenitor. Taken together, the transcriptomic identification, prospective isolation. clonal in vitro differentiation assays, and in vivo engraftment results offer strong evidence for the existence of a functionally bipotent glial progenitor in the fetal human brain.

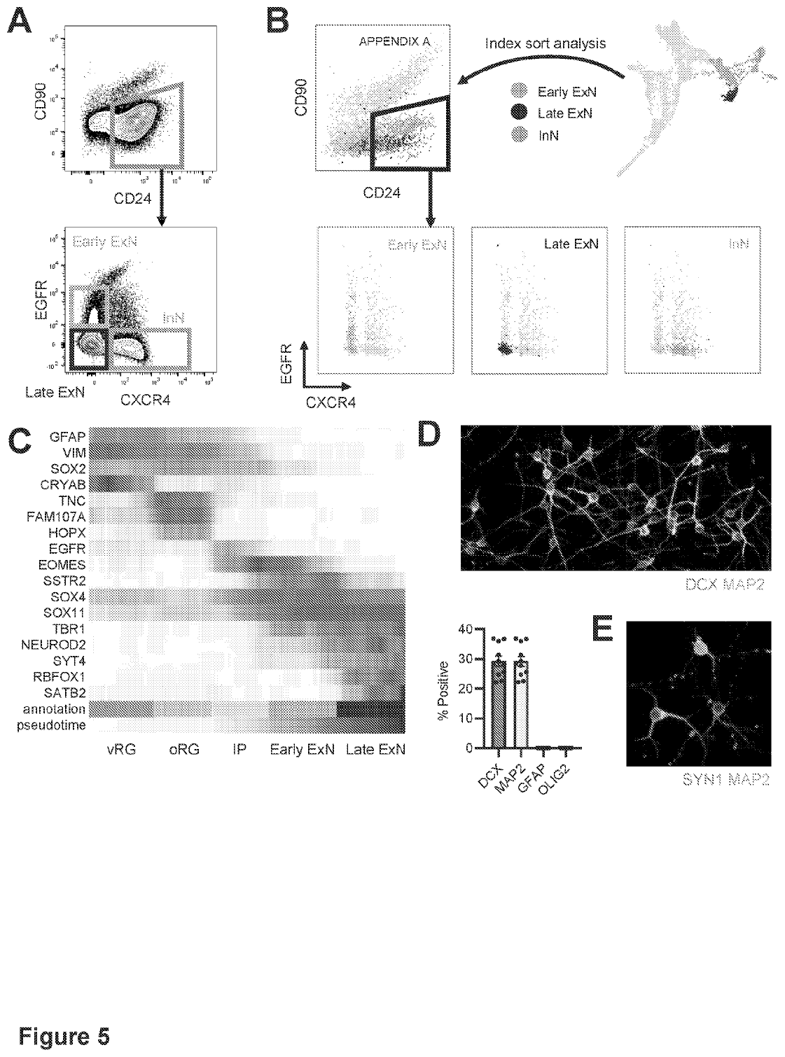

CD24⁺THY1^{-/lo} expression identifies neuronal precursors. Index-sort analysis showed that neuron precursors, broadly expressing DCX, SOX4, and SOX11 transcripts, were highly enriched within the CD24⁻THY1^{-/lo} gate, with further immunophenotypic heterogeneity observed with respect to EGFR and CXCR4 expression (FIG. 5A). Within the CD24⁺THY1^{-/lo} gate, the EGFR⁺CXCR4⁻ gate enriched for early glutamatergic excitatory neuron (ExN) lineage cells, the EGFR⁻CXCR4⁻ gate enriched for later ExN, while the EGFR⁻CXCR4⁺ gate enriched for GABAergic inhibitory neuron lineage cells (InN) (FIG. 5B). These markers are consistent with known biology, as interneurons are known to migrate towards gradients of CXCL12 via interactions with its receptor CXCR4. In particular, during early fetal brain development, the ganglionic eminences give rise to InN precursors that undergo a long-range tangential migration to reach the cortex. Pseudotime analysis confirmed mRNA expression changes along the neuron maturation trajectory, with intermediate progenitor genes (EOMES) preceding early (NEUROD2) and late (SATB2) neuron markers (FIG. 5C).

To assess their functional behavior in vitro, we then sorted CD24⁺THY1^{-/lo}CXCR4⁻ cells for culture, whereupon they differentiated into a homogeneous population of DCX⁺MAP2⁺ cells with neuronal morphology (FIG. 5D). Synapsin-1 (SYN1) puncta were observed between the neurons, suggesting the formation of synaptic connections. No GFAP⁺ or OLIG2⁺ cells were observed, suggesting that these cells are indeed committed to a neuronal fate. CD24⁺THY1^{-/lo}CXCR4⁺ InN could not be successfully cultured. We attempted to transplant both ExN and InN into mice, but observed no engraftment for either, even when transplanting up to 2 million cells. This result likely reflects the cells being too mature to robustly engraft. This finding may have implications for neuronal transplantation studies for applications in regenerative medicine.

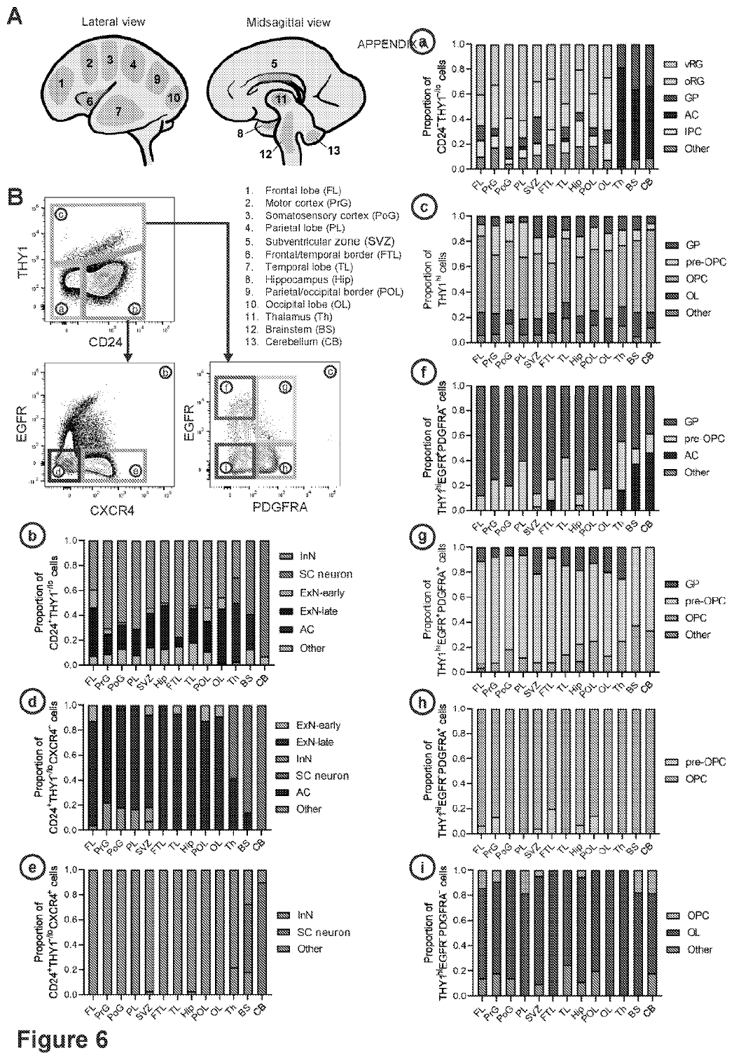

NSPC surface markers are conserved across diverse brain regions. Though our studies have been focused on the cortex broadly, we also sought to determine how well our purification scheme applies to specific cortical regions and even subcortical structures. From intact fetal human brain specimens, we dissected out 13 distinct regions: the frontal lobe (FL), motor cortex (precentral gyrus, PrG), somatosensory cortex (postcentral gyrus, PoG), parietal lobe (PL), subventricular zone (SVZ), frontal/temporal lobe border (FTL), temporal lobe (TL), hippocampus (Hip), parietal/occipital lobe border (POL), occipital lobe (OL), thalamus (Th), brainstem (BS), and cerebellum (CB) (FIG. 6A). Each region was separately digested, stained, and index-sorted for Smart-seq3 as above.

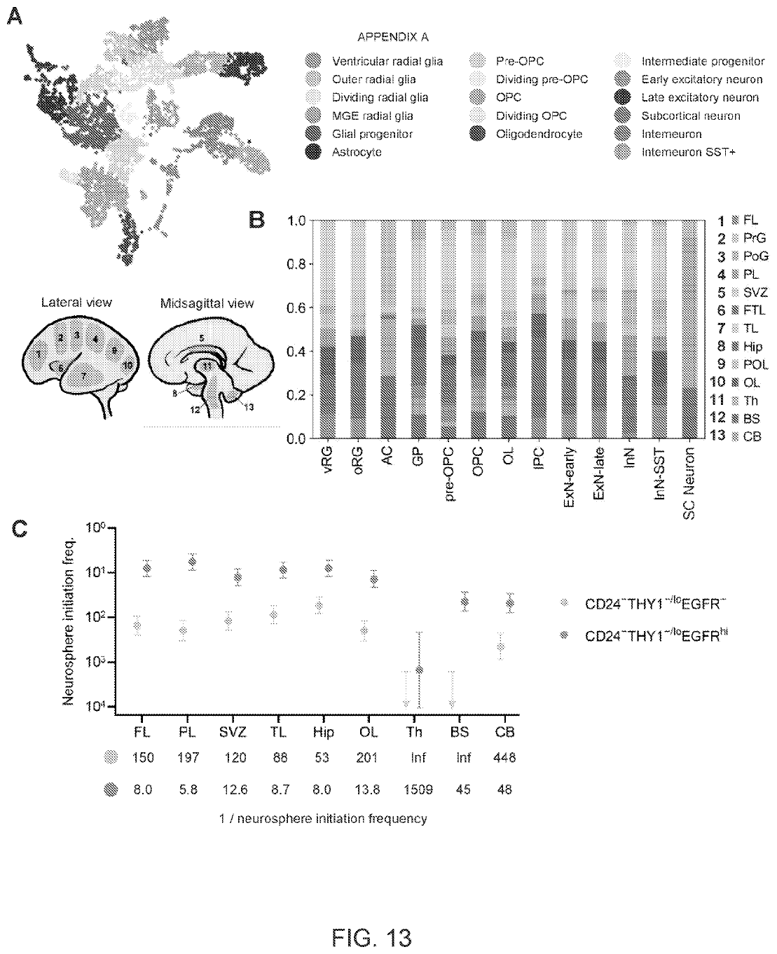

Single cell RNA-seq of the dissected brain regions recovered all lineages described above (FIG. 13A). As expected, ventricular and outer radial glia as well as cortical neuron lineages were represented in all cortical regions, but not the subcortical structures (i.e. thalamus, brainstem, and cerebellum) (FIG. 13B). Glial progenitors, astrocytes, and oligodendrocyte lineages were represented in all brain regions. We also observed a cluster of RELN-expressing neurons found only in the subcortical structures. With this anatomically annotated dataset, we were also able to confirm that even in segmented brain samples (where dissecting out the cortex prior to dissociation was not possible), the vast majority of sequenced cells (86-99%) mapped to a cortical identity. and of the cells that did not map to a cortical identity, nearly all were accounted for by subcortical astrocytes (FIG. 6Ba). Limiting dilution assays from each brain region showed that CD24⁻THY1^{-/lo}EGFR^{hi} cells gave rise to neurospheres at a high frequency in all cortical regions (1 in 5.8 to 1 in 13.8), less so in the brain stem (1 in 45) and cerebellum (1 in 48), and rarely in the thalamus (1 in 1509) (FIG. 13C).

We then performed index-sort analysis to determine how well our previously described purification scheme applies to each brain region. Consistent with our earlier results, the CD24⁻THY1^{-/lo} gate highly enriched for radial glia and astrocytes in all cortical regions; in the subcortical structures, where radial glia are not present, CD24⁻THY1^{-/lo} still enriched for astrocytes (FIG. 6B, a). The CD24⁺THY1^{-/lo} gate was highly specific for neuronal lineages in all brain regions (FIG. 6B, b). CD24⁺THY1^{-/lo}CXCR4⁻ enriched for excitatory neurons in all brain regions. CD24⁺THY1^{-/lo}CXCR4⁺ enriched for interneurons in all cortical regions as well as the thalamus, and, to a lesser extent, the brainstem and cerebellum (FIG. 6B, d-e). The THY1hi gate was highly specific for glial progenitors and oligodendrocyte lineages, regardless of brain region (FIG. 6B, c). The subpopulations within the THY1^{hi} too remained consistent across all brain regions: EGFR^{hi}PDGFRA⁻ enriched glial progenitors, EGFR⁺PDGFRA⁺ enriched pre-OPCs, EGFR⁻PDGFRA⁺ enriched OPCs, and EGFR⁻PDGFRA⁻ enriched mature oligodendrocytes, many to a high degree of purity (FIG. 6B, f-i). Overall, we have shown that our surface markers for NSPCs in the fetal human brain are remarkably conserved even across diverse brain regions, allowing for its broad application to any brain structure at this gestational age of development.

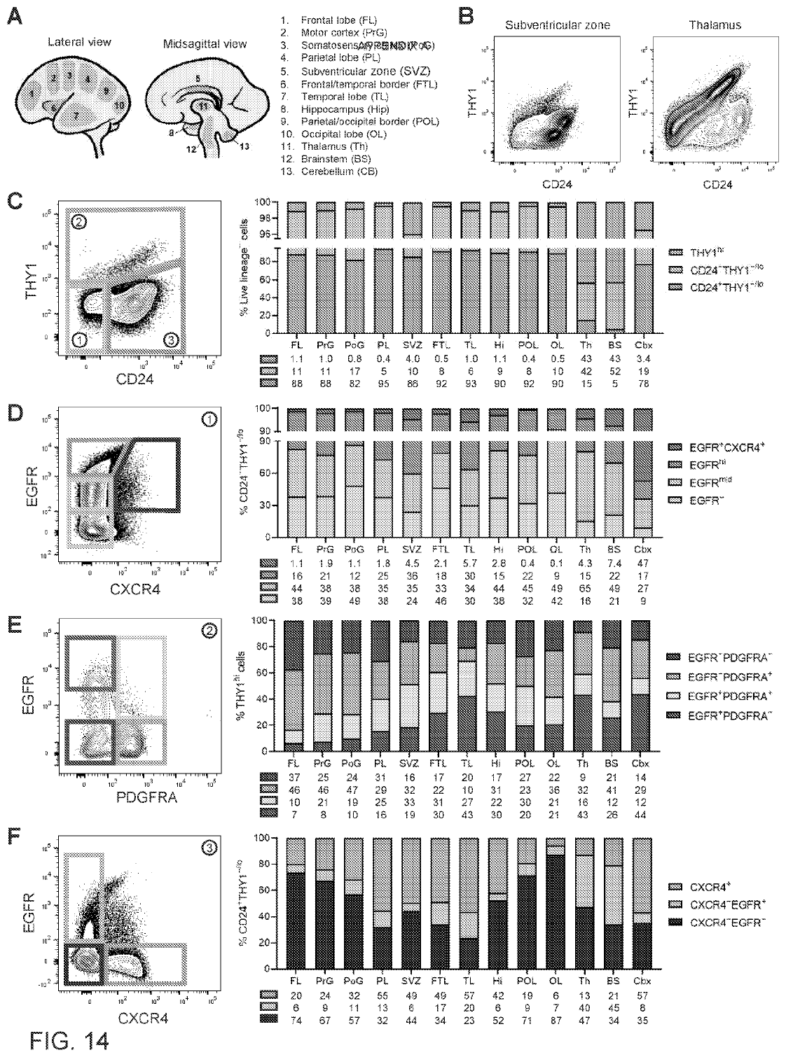

We then used our gating scheme to quantify the frequency of each immunophenotypic population as a percentage of live lineage (blood and vessel) negative cells in all 13 dissected brain regions (FIG. 14). The frequency of THY1hi cells was consistently 0.5-1% in cortical regions, and enriched to 4.0% in the SVZ, possibly reflecting active oligodendrogenesis in the SVZ and OSVZ (FIG. 14B, C). In the thalamus and brainstem, THY^{hi} cells made up over 40% of cells, reflecting the higher extent of myelination in more caudal brain structures at this developmental stage. Our method thus allows for rapid profiling of fetal human brain tissue and their relative cellular makeup via flow cytometry.

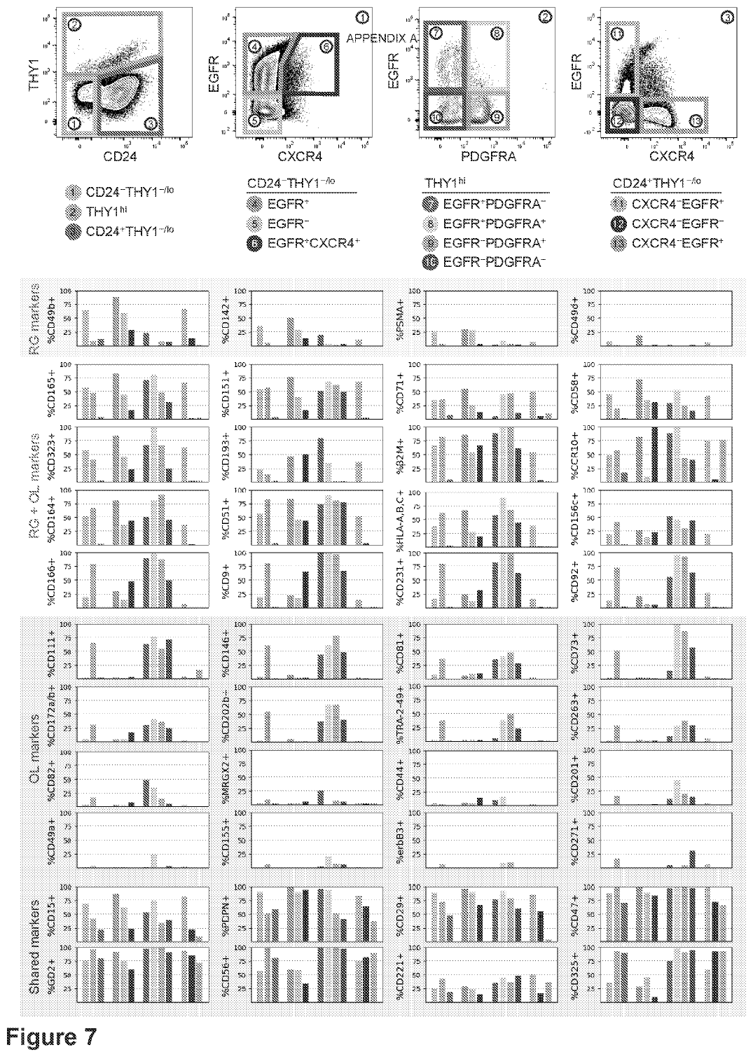

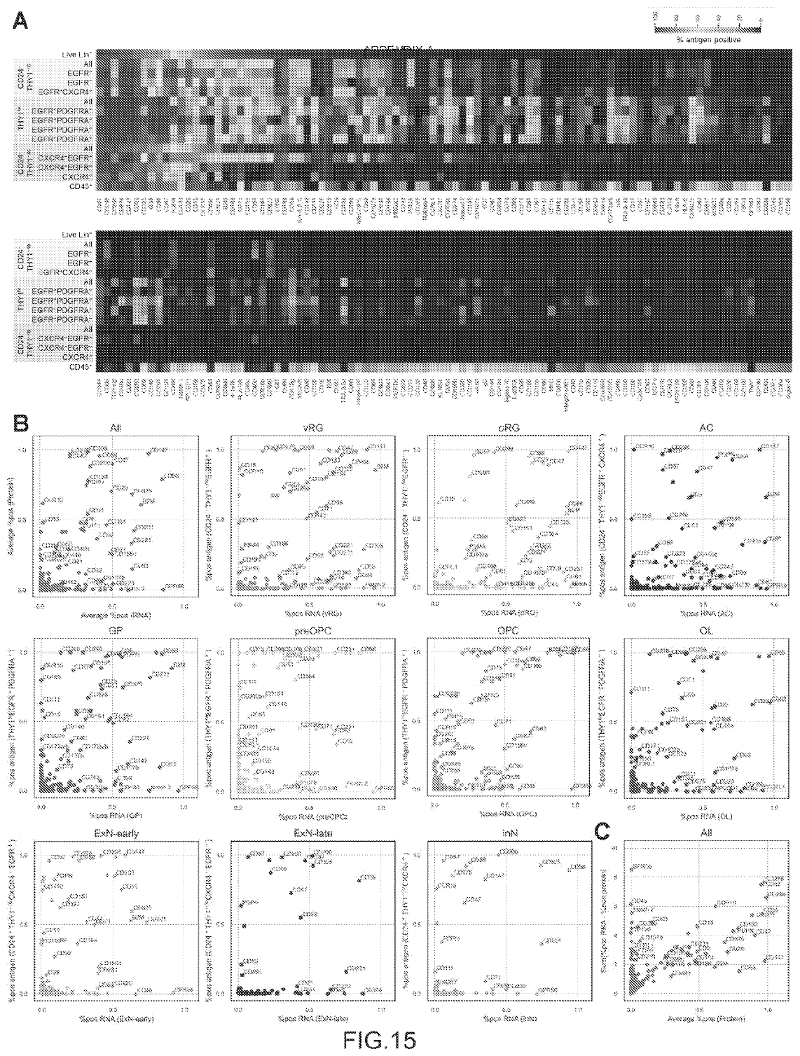

Profiling of the NSPC surfaceome reveals new cell type-specific markers. In addition to our base gating scheme, we measured the expression of 352 additional surface markers using flow cytometry, allowing for cell type-specific profiling of surface antigen expression (FIG. 7, FIG. 15A). We identified several markers specific to CD24⁻THY1^{-/lo} radial glia (CD49b [ITGA2], CD49d [ITGA4], CD142 [F3], PSMA [FOLH1]), as well as those specific to THY1^{hi} oligodendrocyte lineages (CD111 [NECTIN1], CD146 [MCAM], CD81, CD73 [NT5E], CD172a/b [SIRPA/SIRPB], CD202b [TEK]). No single marker was specifically enriched in CD24⁺THY1^{-/lo} neurons, emphasizing the importance of using combinatorial marker expression in isolating pure cell types. Within the CD24⁻THY1^{-/lo} gate, we identify several markers enriched in EGFR⁺ vRG (CD193 [CCR3], CCR10, CD164, CD165, CD51 [ITGAV], CD58), as well as a few enriched in EGFR⁻ ORG (CD325 [CDH2], CD200).

By integrating our transcriptome and surfaceome data, we were able to correlate the percent of cells expressing RNA versus the corresponding surface protein for each marker for each of the identified cell type (FIG. 15B, C). Though some markers showed good correlation between the percent of cells expressing RNA and surface protein (CD56 [NCAM1], CD147 [BSG], 32-microglobulin [B2M], CD29 [ITGB1], CD325 [CDH2], CD164), other markers showed high RNA expression with little surface protein expression (GPR56 [ADGRG1], CD46, Notch2, CD220 [INSR]), or high surface protein expression with little RNA expression (CCR10, CD57 [B3GAT1], CD276, CD298 [ATP1B3]). Thus, though the transcriptomic differences between NSPC types have been previously studied. characterization of surface antigen expression remains essential due to their utility in live sorting, as well as the often-poor correlation between RNA and protein (Supplementary FIG. 1C). Our broad profiling of the NSPC surfaceome thus facilitates future studies on functional heterogeneity within specific cell types, potential molecular mechanisms of lineage restriction, fate specification, migration, and homing in the fetal human brain.

Our data demonstrate that NSPCs can be prospectively isolated from the developing human brain to a high degree of purity based on the expression of defined surface markers. We identify three major neural compartments: CD24⁻THY1^{-/lo} radial glia and astrocytes, THY1^{hi} OPCs, and CD24⁺THY1^{-/lo} neuron precursors, with further immunophenotypic heterogeneity present within each main gate (FIG. 7). These markers are conserved across diverse brain regions. Whereas NSCs maintain multilineage potential, OPCs, astrocytes, and neuron precursors are heavily skewed if not outright committed to their specific lineage. We additionally identify a novel cell type, the glial progenitor, which we found to be lineage-restricted to oligodendroglial and astrocytic fates.

Index-sorting is a powerful tool that bridges the technologies of scRNA-seq (transcriptome) and flow cytometry (immunophenotype), allowing for each sequenced single cell to be mapped back to its original cell-surface profile. We have utilized this method here to rigorously quantify the purity of transcriptomically-defined cell types with respect to their isolation strategy based on combinations of cell-surface markers. With a suitable panel of specific antibodies against cell-surface markers, scRNA-seq with index-sorting is a generalizable method for developing and validating sort strategies in other tissues and cell types of interest. While scRNA-seq, index-sorting, and pseudotime analyses are information-rich methods, they can be thought of as highly sophisticated molecular morphologies. Similar to other morphology-based interpretations, these methods are also insufficient to define lineage relationships. Here we demonstrate that transplantation into the lateral ventricles of neonatal immunodeficient mice can reveal at least some site appropriate activities, perhaps allowing direct testing of lineage differentiation potential.

Previous characterization of NSPCs from the developing human brain has often been done without the ability for prospective isolation. Though these experiments yielded valuable insights on their functional differences, the ability to distinguish these cell types prospectively would be desirable. Immunopanning has been another popular method to purify cell types from the brain, a technique in which specific antibodies are adsorbed to the surface of a dish. Subsequently, cell suspensions are serially incubated in said dishes to enrich or deplete cell types of interest. While this method has been valuable in advancing our understanding of neural cell biology, it has been confounded by nonspecific adsorption of cells to surfaces, and therefore is limited in its ability to precisely and quantitatively isolate pure cell populations based on the expression, especially non-binary expression gradients, of multiple cell-surface markers. It is important to use combinatorial markers, both positive and negative, for isolating truly pure functional NSPC populations.

In vivo transplantation remains the gold standard in stem cell biology to interrogate a cell's developmental potential. Previous transplantation studies of prenatal human NSCs have generally relied on cultured cell lines. This study is the first to orthotopically transplant acutely purified NSPCs from the developing human brain into mice without any culture. Transplanted CD24⁻THY1^{-/lo} cells engrafted and gave rise to astrocytes, oligodendrocytes, and neurons, consistent with their identity as multipotent NSCs. Remarkably, we found that THY1^{hi} cells can also robustly engraft in the mouse brain, and moreover, gave rise exclusively to oligodendrocyte lineages. THY1 is classically thought of as a neuron marker, and its promoter is often used in genetic studies in mice to drive neuron-specific expression. Our findings, however, demonstrate that THY1 in human prenatal brains is in fact a marker of oligodendrocyte lineages, at least during the window of human brain development (GW16-18) under investigation in this study. Our results indicate a nuanced stage-specific variability of marker expression across species and developmental stages.

The existence of a bipotent glial progenitor (GP), giving rise exclusively to oligodendrocytes and astrocytes, has been speculated, but our study is the first to functionally identify this cell type in the developing human cortex. Indeed, our data show that while GPs are A2B5⁺, radial glia, astrocytes, and OPCs are also A2B5⁺ (FIG. 12D). The power to prospectively isolate a cell type is critical for functional studies, especially in human tissues where we lack the usual tools of genetic lineage tracing. Our study is the first to prospectively isolate such a glial progenitor and functionally characterize its bipotent cell fate potential both in vitro and in vivo. We identify ETV4 (ETS Variant Transcription Factor 4) as a GP-specific transcription factor. ETV4 has been associated with the progression of various cancers, including glioblastoma, where its overexpression is a poor prognostic marker. Its role in normal neurodevelopment, however, has not been previously documented. Our work provides both a purification strategy and genetic handle on the glial progenitor, facilitating further investigation on its role in human brain development.

The results presented here demonstrate that distinct cell types from the developing brain can be prospectively isolated based on the expression of multiple surface markers. The combined results from scRNA-seq, in vitro differentiation, and in vivo transplantation offers the most rigorous interrogation of NSPCs' transcriptomic identities and functional behavior. The modular nature of our antibody panel allows for the easy expansion or simplification of the panel based on the cell-type of interest, thus providing a valuable tool for future investigation of both molecular and functional NSPC heterogeneity.

### Example 2

Immunophenotypic definition of neural stem and progenitor cell types. To purify cell-intrinsically distinct putative NSPC fractions, GW16-18 human brain tissue was mechanically and enzymatically dissociated into a single-cell suspension, which was stained with a panel consisting of antibodies against PROM1 (CD133), CD24, THY1 (CD90), CXCR4, EGFR, PDGFRA, and non-neural lineages (CD45, PECAM1[CD31], CD34, ENG [CD105], and GYPA [CD235a]). The stained cell suspension was analyzed using fluorescence-activated cell sorting (FACS); the observed immunophenotypic profile was extremely consistent across over a dozen samples of this gestational age group. Various populations that stratified based on cell-surface marker expression (immunophenotype) were index-sorted into 96-well plates for full-length single-cell RNA-sequencing (scRNA-seq) using Smart-seq2 or Smart-seq3. Index-sorting records the fluorescence intensities for each sorted single cell, allowing for direct mapping between surface marker profile and the expressed transcriptome.

Unsupervised clustering of single-cell transcriptomes identified various putative NSPC populations, which were annotated using input from a combination of functional assays and expressed mRNAs of known marker genes. These transcriptomically-defined cell types include putative ventricular radial glia (vRG). outer radial glia (ORG), astrocytes (AC), pre-oligodendrocyte precursor cells (pre-OPC), OPCs, oligodendrocytes (OLs), intermediate progenitor cells (IPCs), and excitatory and inhibitory neuronal lineage cells (ExN, InN) (see details below). Index-sort data allowed for each individual sequenced cell to be mapped back to its original immunophenotype. RNA and cell-surface protein expression did not always correlate. most notably with the glycosylphosphatidylinositol (GPI)-anchored surface molecules THY1 and CD24. Using the index-sort information, the putative NSCs were found to be CD24−THY1−/lo, the putative OPCs were THY1hi, and the putative neuronal lineage cells were CD24+THY1−/lo, with additional heterogeneity present in each compartment. It was previously reported that CD133+CD24-cells are highly enriched for NSCs; indeed, the index-sort analysis confirms that this gate largely consists of vRG. The gating strategy resulted in remarkably pure populations of sorted cells as validated by scRNA-seq, allowing for prospective isolation of various putative NSPCs for downstream functional assays.

CD24−THY1−/lo expression identifies phenotypic NSCs. Index-sort data showed that putative NSCs, broadly expressing SOX2, GFAP, and VIM transcripts, were enriched by the CD24−THY1−/lo gate, with further immunophenotypic heterogeneity observed in cell-surface expression of EGFR and CXCR4. Within the CD24−THY1−/lo gate, the EGFRhi population was enriched for putative vRG (expressing CRYAB and FBXO32 transcripts). while the EGFR− population was enriched for putative oRG (expressing HOPX and LIFR transcripts, and LIFR protein). A population of EGFR+CXCR4+ putative astrocyte (AC) lineage enriched for PAX3 and EDNRB transcripts was observed. Intracellular staining followed by flow cytometry confirmed that, similar to their expression at the transcript level, the CD24−THY1−/lo cells also highly expressed the protein versions of known NSC marker genes, including SOX1, SOX2, GFAP, and PAX6. Notably, the expression of PAX3 transcript was restricted to the putative AC lineage, suggesting that PAX3 may have a role in early lineage bifurcation between the NSC and AC lineages. This finding is in contrast with studies in mouse showing that Pax3 downregulation is associated with differentiation towards an astrocytic fate. It is possible that the AC population represents an early progenitor population where PAX3 induction is initially required before being downregulated with maturation. Alternatively, there could be considerable cellular heterogeneity within the AC lineage with respect to PAX3 expression in human prenatal brains.

To assess whether the putative NSC subsets are functional, their self-renewal and differentiation capabilities in vitro and in vivo was assessed. It was previously shown that CD133+CD24− cells can self-renew and be maintained in the presence of LIF, FGF, and EGFR and expanded as neurospheres. Consistent with the previous results, most of the CD24−THY1−/lo putative NSCs correspond to the CD133+CD24− cells, and readily formed neurospheres when cultured. In addition, the in vitro limiting dilution assays showed that within the CD24−THY1−/lo gate, the EGFRhi population possesses markedly higher neurosphere initiation frequency (1 in 4.5) compared to the EGFR− population (1 in 10⁴)—a difference that was not attributable to cell cycle. To assess the functional behavior of individual CD24−THY1−/lo putative NSCs in vitro, clonal neurospheres were generated from single sorted cells. Individual single-cell-derived clonal neurospheres were dissociated and were subjected to differentiation conditions. Both EGFR− and EGFRhi NSC clones could be differentiated into DCX+, GFAP+, and rare O4+ cells, demonstrating their competence to give rise to neuron, astrocyte, and oligodendrocyte lineages. In contrast, EGFR+CXCR4+ cells gave rise almost exclusively to GFAP+ cells, suggesting they are largely committed to an astrocytic fate.

To functionally characterize the putative NSCs in vivo, acutely isolated cells were transplanted directly, without intervening culture, into the lateral ventricles of neonatal NOD-scid-IL2Rgnull (NSG) immunodeficient mice. After 6 months, the level of engraftment was assessed by immunofluorescent (IF) staining for human-specific antigens. Transplanted NSCs migrated and engrafted extensively throughout the brain and differentiated to give rise to all three major neural lineages: GFAP+ astrocytes/NSCs, OLIG2+ oligodendrocytes, and NeuN+ or MAP2+ neurons. Remarkably, the CD24−THY1−/loEGFRhi NSCs resulted in significantly higher levels of engraftment compared to CD24−THY1−/loEGFR−NSCs, with 1.8-fold more OLIG2+ cells and 2.3-fold more GFAP+ cells observed across four replicates. Collectively, the in vitro and in vivo results confirm that the putative NSC fractions are indeed functional with respect to their self-renewal capability and differentiation potential. In addition, among the CD24−THY1−/lo NSCs, EGFRhi cells are likely enriched for relatively primitive multipotent NSCs compared to EGFR− cells.

THY1hi expression identifies oligodendrocyte lineages. Index-sort data combined with unsupervised clustering showed that the putative oligodendrocyte lineages, broadly expressing OLIG1, OLIG2, and SOX10 transcripts, was enriched by the THY1hi gate. This gate highly enriched the OL lineages, suggesting THY1 to be a pan-oligodendrocyte lineage marker at least at this developmental stage. Among the THY1hi cells, additional heterogeneity was observed with respect to cell-surface expression of EGFR and PDGFRA, resulting in four immunophenotypic populations: THY1hiEGFR+PDGFRA− (ThiE+P−), THY1hiEGFR+PDGFRA+ (ThiE+P+), THY1hiEGFR−PDGFRA+ (ThiE−P+), and THY1hiEGFR−PDGFRA− (ThiE−P−). Index-sort analysis showed that the ThiE+P− gate represents a heterogeneous population likely consisting of both putative OPCs and NSCs. The other gates allowed isolation of much purer populations, with the ThiE+P+ gate highly enriched for putative pre-OPCs (expressing EGFR, MKI67, and PCNA transcripts), the ThiE−P+ gate for putative OPCs (expressing PDGFRA and PCDH15 transcripts), and the ThiE−P− gate for relatively more mature oligodendrocytes (expressing MYRF and MBP transcripts). Consistent with the gene expression-based inference, pseudotime analysis also recapitulated the oligodendrocyte maturation trajectory. Plotting the pseudotime by immunophenotype shows a gradient of maturation from the ThiE+P+ to the ThiE−P+, and then to the ThiE−P− gates. Indeed, the ThiE+P+ gate was enriched for actively-cycling cells, suggesting that they are a transit-amplifying population that expands the OPC pool, likely corresponding to previously-described pre-OPCs.

The functional behavior of the putative OPC subsets was next assessed in vitro. Limiting dilution assays showed that the ThiE+P− and the ThiE+P+ cells had a high neurosphere initiation rate (1 in 9.8 and 1 in 5.6, respectively), in contrast to the ThiE−P+ and the ThiE−P− cells (1 in 872 and 1 in 1634, respectively), further supporting the notion that EGFR expression marks a more proliferative pre-OPC subtype. It was previously reported that the generation of neurospheres from the CD133+CD24-population, which, from the index-sort data in the current study, contain both vRG and pre-OPC neurosphere-initiating cells (FIG. d). In this study, by probing for additional cell-surface markers, this heterogeneity was resolved and report two possibly related yet distinct neurosphere-initiating cell subsets: the CD24−THY1−/loEGFR+ (NSC) and the THY1hiEGFR+PDGFRA+ (OPC) subsets. Thus, both the NSC and the OPC neurospheres derived in this study represent purer and cell-intrinsically distinct populations.

Next, each of the four OPC populations were bulk sorted and cultured them in the absence of growth factors to induce differentiation. Subsequent IF imaging of resulting cells post-differentiation revealed O4+ cells with distinctive oligodendrocytic morphology. When the ThiE+P−, ThE+P+, and ThiE−P+ populations were subjected to the in vitro differentiation, the resulting proportions of O4+ cells showed an increasing trend from 2.3% to 9.6%, and then to 83.5%, respectively. The ThiE−P− cells did not survive well in culture likely due to their more mature state-nevertheless. the survivors still gave rise to 61.6% O4+ cells in the culture conditions. These results provide supportive evidence, though not conclusive proof, for the maturation trajectory predicted by the pseudotime analysis described above. Strikingly, no other populations gave rise to any appreciable numbers of O4+ cells.

To assess their functional behavior in vivo, each of the four THY1hi OPC subsets was transplanted into the lateral ventricles of neonatal NSG mice. Three of the subsets, ThiE+P−, ThiE+P+, and ThiE−P+, engrafted robustly in the brain. Cells sorted from the ThiE+P− gate gave rise to both GFAP+ and OLIG2+ human cells, consistent with the index-sort data, which had suggested it to be a heterogeneous population. Remarkably, cells sorted using the ThiE+P+ and ThiE−P+ gates gave rise only to OLIG2+ cells, with no observed GFAP+ or NeuN/MAP2+ cells, suggesting that these cells are indeed committed to an oligodendrocytic fate. The T^{hi}E-P-cells did not engraft, possibly because they are too mature and post-mitotic. In summary, the collective in vitro and in vivo results confirm that the purified OPC subsets are engraftable and are indeed lineage committed to form only oligodendrocytes.

CD24+THY1−/lo expression identifies neuronal precursors. Index-sort analysis showed that neuron precursors, broadly expressing DCX, SOX4, and SOX11 transcripts, were highly enriched within the CD24+THY1−/lo gate, with further immunophenotypic heterogeneity observed with respect to EGFR and CXCR4 expression. Within the CD24+THY1−/lo gate, the EGFR+CXCR4− gate enriched for early excitatory neuron (ExN) lineage cells. the EGFR−CXCR4− gate enriched for later ExN, while the EGFR−CXCR4+ gate enriched for GABAergic inhibitory neuron lineage cells (InN). These markers are consistent with known biology, as interneurons are known to migrate towards gradients of CXCL12 via interactions with its receptor CXCR4. In particular, during early fetal brain development, the medial ganglionic eminence gives rise to InN precursors that undergo a long-range tangential migration to reach the cortex. Pseudotime analysis confirmed mRNA expression changes along the neuron maturation trajectory, with intermediate progenitor genes (EOMES) preceding early (NEUROD2) and late (SATB2) neuron markers.

To assess their functional behavior in vitro, CD24+THY1−/loCXCR4-cells were sorted for culture, whereupon they differentiated into a homogeneous population of DCX+MAP2+ cells with neuronal morphology. Synapsin-1 (SYN1) puncta were observed between the neurons, suggesting the formation of synaptic connections. No GFAP⁺ or O4+ cells were observed, suggesting that these cells are indeed committed to a neuronal fate. CD24+THY1−/loCXCR4+ InN could not be successfully cultured. Both ExN and InN were attempted to be transplanted into mice, but observed no engraftment for either, even when transplanting up to 2 million cells. This result likely reflects the cells being too mature to robustly engraft. This finding may have implications for neuronal transplantation studies for applications in regenerative medicine and could explain previous largely unsuccessful attempts to treat severe Parkinson's disease by transplanting embryonic dopaminergic neurons.

### Methods

Mice. NSG (NOD.Cg-Prkdc^{scid} Il2rg^{tm1Wjl}/SzJ, JAX: 005557) immunodeficient mouse strains were used as recipients for transplantation of human NSPCs. All mice were transplanted between postnatal day 1-2 and were sacrificed at 6 months post-injection. Animals were housed in Stanford University's animal core facility strictly in accordance with National Institute of Health (NIH) and Stanford's Administrative Panel on Laboratory Animal Care (APLAC) guidelines.

Fetal brain samples. Human fetal brain samples were obtained from Advanced Bioscience Resources (ABR, Alameda, CA) and shipped overnight in BioWhittaker RPMI-1640 media supplemented with L-glutamine. Samples ranged in age from 18 to 20 weeks of gestation (GW17-19 with no restrictions on race or gender. No Institutional Review Board (IRB) approval was required for procurement of deidentified samples from ABR as per Stanford University guidelines. All subsequent in vitro and in vivo experiments that utilized cells from fetal brain tissues thus obtained were performed strictly as per pre-approved guidelines set by Stem Cell Research Oversight (SCRO) (SCRO protocol #735 and APLAC protocol #26209) at Stanford university.

Human fetal brain dissociation. Deidentified prenatal human brain samples were obtained from Advanced Bioscience Resources (Newark, California) and shipped overnight. The age range for samples was 16 to 18 gestational weeks with no restrictions on race or sex. Prenatal brain sample procurement and handling was done in accordance with guidelines set by the Stanford Institutional Review Board (IRB) and the Stanford Stem Cell Research Oversight (SCRO) Panel. Intact samples were dissected into distinct anatomical regions by licensed neuropathologists. Samples were gently chopped with a razor blade and resuspended in digestion buffer A, which consists of Hank's balanced salt solution (HBSS) (Thermo. cat. 24020117) with 10 μg/mL Liberase (Roche, cat. #5401119001) and 200 g/mL DNase I (Worthington, cat. #LS002007). The sample was then incubated twice at 37° C. under constant agitation for 40 minutes. Afterwards, cells were spun down and resuspended in digestion buffer B, consisting of Accutase (Innovative Cell Technologies, cat. #AT104) supplemented with 200 μg/mL DNase I, then incubated at 37° C. for 15 minutes under constant agitation. Red blood cell removal was performed using a density gradient by layering the cell suspension on top of Histopaque (Sigma, cat. #10771) at a 2:1 ratio, then centrifuging at 400×g for 30 minutes at 25° C. with low acceleration and no brakes. The buffy coat at the fluid interface was collected, washed, and counted before staining with fluorochrome-conjugated antibodies. All washes were performed in HBSS supplemented with 0.1% polyvinyl alcohol (PVA) (Sigma, cat. #P8136).

Fluorescence-activated cell sorting (FACS). Brain tissue was dissociated into a single cell suspension as described above in the “Human fetal brain dissociation” section. The cell suspension was stained with fluorochrome-conjugated antibodies against CD133 (clone13H2), CD24 (clone 32D12, Miltenyi, cat. #130-095-954), CD90 (clone 5E10, Biolegend, cat. #328108), CXCR4 (clone 12G5, Biolegend, cat. #306528), EGFR (clone AY13, Biolegend, cat. #352910), PDGFRA (clone 16A1, Biolegend, cat. #323504), CD31 (clone WM59, BD Biosciences, cat. #563652), CD34 (clone 581, BD Biosciences, cat. #562383), CD45 (clone HI30, Biolegend, cat. #304010), CD105 (clone 266, BD Biosciences, cat. #562380). and CD235a (clone HIR2, Biolegend, cat. #306606), all at a dilution of 1:50. Propidium iodide (PI) (Sigma, cat. P4170) was added as a viability marker immediately prior to analysis (1 μg/mL). Flow cytometry was performed on a FACS Aria II (BD Biosciences). Debris was excluded using forward and side scatter area, and doublets were excluded using a stringent two-step gating based on forward and side scatter height versus width. Gating schemes were guided by fluorescence-minus-one (FMO) controls. The highest purity setting (“single cell”) was used to collect cells for scRNA-seq and limiting dilution neurosphere initiation assays. The “4-way purity” setting was used for bulk sorting of cells for in vivo transplant and in vitro differentiation assays.

Surfaceome profiling of human fetal brain cells. Brain tissue was dissociated into a single cell suspension and prepared for flow cytometry as described above in the “Human fetal brain dissociation.” Cells were stained with the antibody panel described above in “Fluorescence activated cell sorting (FACS),” omitting the antibody against CD133 to free up the PE channel. Cells were screened for 352 additional surface markers using the LEGENDScreen™ Human PE Kit (Biolegend, cat. #700007) per manufacturer protocol. Cells were analyzed on a BD FACSymphony A5 in 96-well plate format. An average of 130,000 events were recorded for each marker. The percentage of cells staining positive for each marker was determined for the corresponding parent gate using the appropriate isotype control.

For the comparison between antigen and RNA expression, each surface marker on the LEGENDScreen panel was matched to its corresponding gene. Glycan antigens were matched to the gene responsible for their synthesis (e.g. FUT4 for CD15, B3GAT1 for CD57). The percentage of antigen-positive cells within each immunophenotypic gate was correlated with the percentage of RNA-expressing cells within the corresponding transcriptomic cluster for that gate.

Single cell RNA sequencing (scRNA-seq). Single cell capture and RNA extraction. Brain tissue was dissociated into a single cell suspension and prepared for FACS as described above in the “Human fetal brain dissociation” and “Fluorescence activated cell sorting (FACS)” sections above. Single cells were index-sorted into 96-well plates containing 2 μL lysis buffer (1 U/μL RNase inhibitor (Clontech, cat. #2313B), 0.1% Triton (Thermo Fisher Scientific, cat. #85111), 2.5 mM dNTP (Thermo Fisher Scientific, cat. #10297018), 2.5 μM oligo dT₃₀VN (Integrated DNA Technologies, 5′-AAGCAGTGGTATCAACGCAGAGTACT₃₀ VN-3′), and 1:600,000 ERCC (external RNA controls consortium) RNA spike-in mix at 1:600,000 (Thermo Fisher Scientific, cat. #4456739) in UltraPure water (Thermo Fisher Scientific, cat. #10977015) (Picelli et al., 2014). Immediately after sorting, plates were centrifuged at 3000×g for 30 seconds at 4° C., snap frozen on dry ice, and stored at −80° C.

Reverse transcription and pre-amplification. Reverse transcription (RT) and cDNA pre-amplification was performed using the Smart-seq3 protocol with minor modifications (Hagemann-Jensen et al., 2020). In brief, lysis plates were thawed on ice and incubated at 72° C. for 3 minutes then immediately snap chilled on ice to anneal the oligo dT 30 VN primer. For reverse transcription, 3 μL of RT mix (25 mM Tris-HCl pH 8.5 (Teknova, cat. #T5085), 0.5 U/L RNase inhibitor (Clontech, cat. #2313B), 8 mM dithiothreitol (DTT) (Promega, cat. #P1171), 30 mM NaCl (Thermo Fisher Scientific, cat. #AM9760G), 2.5 mM MgCl₂ (Thermo Fisher Scientific, cat. #AM9530G), 1 mM GTP (Thermo Fisher Scientific, cat. #R0461), 2μM TSO (Integrated DNA Technologies, 5′ AAGCAGTGGTATCAACGCAGAGTGAATrGrGrG-3′), 5% polyethylene glycol (Sigma, cat. #P1458), and 2 U/μL Maxima H-minus reverse transcriptase (Thermo Fisher Scientific, cat. #EP0753) in UltraPure water) was added to each well using a Mantis liquid handler (Formulatrix), and incubated in a C1000 Touch Thermal Cycler (BioRad) at 42° C. for 90 min, and then 70° C. for 15 min to terminate the reaction. Afterwards for preamplification, 7.5 μL of PCR mix (1.67X KAPA HiFi HotStart ReadyMix (Kapa Biosystems, cat. #KK2602) and 0.17 μM IS PCR primer (Integrated DNA Technologies, 5′-AAGCAGTGGTATCAACGCAGAGT-3′) in UltraPure water) was added to each well and cycled using the following program: (1) 98° C. for 3 min, (2) denaturing at 98° C. for 20 sec, (3) annealing at 67° C. for 15 sec, (4) elongation at 72° C. for 6 minutes, (5) repeat from step 2 24 times, and (6) 72° C. for 5 minutes. Preamplified cDNA was then purified using 0.65-0.75X volume of calibrated AMPure XP beads (Beckman Coulter, cat. #A63882) to remove residual reaction components and oligos smaller than 400 base pairs, and eluted in 12.5 μL UltraPure water.

Quality control. From the purified cDNA, 1 μL was taken for quality control for each well. cDNA concentration and size distribution for each well was determined on a capillary electrophoresis-based Fragment Analyzer (Advanced Analytical). Wells with a concentration less than 1.7 ng/μL were excluded; this cutoff was determined by measuring the concentration blank wells with ERCC but no sorted cell. The wells within the 96 well plates with cDNA concentration above the cutoff value were then consolidated and reformatted to a new 384 well plate using the Mosquito X1 liquid handler (SPT Labtech), such that in the destination 384 well plate each well's concentration was also normalized to a desirable concentration range of 1.7-4.0 ng/μL by diluting with UltraPure water.

Library preparation and sequencing. Normalized cDNA was used to prepare Illumina sequencing libraries. Tagmentation was performed by combining 0.4 μL cDNA with 1.2 μL homebrew Tn5 mix consisting of 1 ng/μL Tn5 enzyme, 16 mM Tris-HCl PH 7.6, 16 mM MgCl₂ (Thermo Fisher Scientific, cat. #AM9530G) and 8% dimethylformamide (DMF) (Thermo Fisher Scientific, cat. #AC327171000) in UltraPure water. The reaction was stopped by adding 0.4 μL neutralization buffer (0.1% SDS). Indexing PCR reactions were performed by adding 0.4 μL of 5 μM i5 indexing primer, 0.4 μL of 5 UM i7 indexing primer (Integrated DNA Technologies, custom made 7680-plex unique dual index-primer set), and 1.2 μL KAPA HiFi HotStart ReadyMix (Kapa Biosystems, cat. #KK2602). PCR amplification was performed on a C1000 Touch Thermal Cycler (BioRad) using the following program: (1) 72° C. for 3 min, (2) 95° C. for 30 sec, (3) denaturing at 98° C. for 10 sec, (4) annealing at 67° C. for 30 sec, (5) elongation at 72° C. for 60 sec, (6) repeat from step 3 10 times. For each 384 well plate, 1 μL was taken from each well for pooling, followed by purification using 0.8X volume of AMPure XP beads (Beckman Coulter, cat. #A63882). The 384-cell library pool from each plate was analyzed for concentration and size distribution using Fragment Analyzer (Advanced Analytical). Twenty 384-cell library pools were then normalized for concentration, further pooled to get a 7680-plex library pool, which was purified once more and concentrated using 0.8X AMPure beads. The final 7680-plex library pool was then sequenced on a NovaSeq 6000 S4 flow cell (Illumina) to obtain ˜1-2 million 2×150 base-pair paired-end reads per cell.

Computational Methods. Read mapping. Sequences were demultiplexed using bcl2fastq version 2.19.0.316. 3′ adapter sequences were removed from reads using skewer v0.2.2 (Jiang et al., 2014), and aligned to the hg38 genome (Gencode version GRCh38.p13) with STAR aligner version 2.6.1d using 2-pass mapping (Dobin et al., 2013). Briefly, as a first pass, reads for every cell were aligned using STAR genome index generated using the Gencode transcript annotation for the human genome (version 34). Mapped splice junctions for each cell from the first-pass mapping were extracted, aggregated together and a new STAR index was created where any newly discovered splice junctions were included in addition to the existing Gencode annotation during genome index generation. The new STAR index with all known and newly identified splice-junctions was then used for second pass read mapping. Parameters used for STAR mapping were adapted from the ENCODE long-mRNA-pipeline (https://github.com/ENCODE-DCC/long-read-rna-pipeline) recommendations, also detailed in the STAR manual. In addition to the ENCODE recommended options we also used the “—quantMode TranscriptomeSAM” option during second-pass mapping to generate a bam file containing a catalog of all reads mapped to the transcriptome. This bam file was used as input to calculate expression levels of either genes (sum total of expression levels of all known transcript variants) or individual transcripts using RSEM version 1.3.3 with settings “—single-cell-prior”, which accounts for the sparse nature of mRNA detection usually prevalent in scRNA-seq (Li and Dewey, 2011).

Data preprocessing. Gene count tables were combined with metadata using the Scanpy python package v.1.8.2 (Wolf et al., 2018). We filtered out genes expressed in fewer than 3 cells, as well as cells with fewer than 500 detected genes or 5000 read counts. The data were normalized using size factor normalization so that every cell has 10,000 read counts, log transformed, and scaled to a maximum value of 10. Highly-variable genes were computed using default parameters. We then performed principle component analysis, computed the neighborhood graph, and clustered the data using the Leiden method (Traag et al., 2019). PAGA was used to visualize data, as well as reconstruct gene expression changes along maturation trajectories (Wolf et al., 2019). A total of 9,454 cells across four donors were included in the final analysis, with an average gene count of 3962. Step-by-step instructions to reproduce preprocessing and analysis of data are available on GitHub.

k-nearest neighbor classifier. To determine the anatomical origin of cells sequenced from segmented samples (where dissection of the cortex prior to dissociation was not possible), we implemented a k-nearest neighbor classifier, using our intact sample for ground truth anatomical labels. Each cell of unknown anatomical origin was classified to a cortical or subcortical identity based on a plurality vote of its k most similar neighbors of known anatomical origin (k=5).

In vivo transplantation of NSPCs. NSPCs were purified using FACS and resuspended in 2 μL HBSS with Fast Green dye (Sigma, cat. F7252) for better visualization of the injected site. Neonatal mice (postnatal day 1) were anesthetized using hypothermia and placed on a stereotaxic device (Harvard Apparatus) fitted with a mouse neonate adaptor (Cunningham). Light illumination was used to identify the sinus above Lambda as the reference point. Burr holes were made in the skull cartilage at the injection site using a 30-gauge needle. The cell suspension was injected using a Hamilton syringe with a 33-gauge needle into the lateral ventricles using the Micro4 microsyringe pump controller (World Prevision Instruments) at a rate of 1 μL/min, with 1 μL injected per side. Successful injection into the ventricles was confirmed visually with light illumination. The following coordinates were used for transplant; anteroposterior from midline (A), lateral from midline (L), ventral from surface of brain (V). Lateral ventricles (A, L, V)=(0.8, +1.5, 2.0) mm with reference to lambda. All in vivo experiments described in this section that utilized cells from fetal brain tissues were performed strictly as per pre-approved guidelines set by Stem Cell Research Oversight (SCRO) at Stanford university (SCRO protocol #735; also see above).

Cell culture. To generate neurospheres, acutely isolated NSPCs were cultured at a density of 10⁵ cells/mL in fetal growth media (FGM) consisting of X-VIVO 15 media (Lonza, cat. #O4-744Q) supplemented with N-2 (Thermo Fisher Scientific, cat. #17502048), heparin (STEMCELL Technologies, cat. #07980), N-acetylcysteine (VWR, cat. #E-3710), 20 ng/ml fibroblast growth factor 2 (FGF2) (Shenandoah Biotechnology, cat. #100-146), 20 ng/ml epidermal growth factor (EGF) (Shenandoah Biotechnology, cat. #100-26), and 10 ng/ml leukemia inhibitory factor (LIF) (Sigma, cat. #LIF1010).

Limiting dilution neurosphere initiation assay. Limiting dilution assays were conducted to quantify the frequency of cells in a population that initiate neurospheres. Known numbers of primary NSPCs were sorted at single cell purity into 96 well plates containing 100 μL of FGM (see above). Half of the media was replaced with fresh media every week. Plates were scored at 4 weeks, with wells containing at least one neurosphere being considered positive. Linear regression analysis of the proportion of positive wells at each cell concentration was used to determine the neurosphere initiation frequency (Hu and Smyth, 2009).

In vitro differentiation of cells. For differentiation assays, primary NSPC populations were bulk-sorted on the 4-way purity setting, and directly plated into 96 well plates coated for 12 hours with poly-L-ornithine hydrobromide (Millipore Sigma, cat. #P3655) and laminin mouse protein (ThermoFisher Scientific, cat. #23017015) in differentiation media consisting of DMEM/F12 (ThermoFisher Scientific, cat. #11320082), heparin, N-acetylcysteine, N-2, and B27 (ThermoFisher Scientific, cat. #17504044), but no other growth factors or cytokines. For clonal differentiation assays, we index-sorted single CD24⁻CD90⁻ cells that were either EGFR⁻ or EGFR^{hi} into 96 well plates containing FGM, and cultured them for 4 weeks to obtain clonally-derived neurospheres. Single cell-derived individual neurospheres were then dissociated, and either reanalyzed via flow cytometry or plated onto polyornithine/laminin-coated wells in differentiation media. After 4 days of in vitro culture, cells were fixed using 4% paraformaldehyde (PFA), and then stained with antibodies for immunofluorescence (IF).

Histology. Mice were sacrificed 6 months post-transplant and perfused using phosphate buffered saline (PBS) supplemented with 20 mM EDTA. The brain was dissected out and fixed in freshly prepared 4% paraformaldehyde (PFA) solution in PBS (Electron Microscopy Sciences, cat. #15710) for 16 hours and then transferred to 30% sucrose solution for cryoprotection. Afterwards, 40 μm sagittal sections of the brains were sliced on a Leica SM2010 R Sliding Microtome. Tissue slices were kept as floating sections in PBS at 4° C. until ready for staining.

For fetal human brain sections, cortical tissues were fixed in 4% PFA for 16 hours, transferred to 30% sucrose solution for cryoprotection, then embedded in optimal cutting temperature (OCT) compound (Tissue-Tek®) and frozen at −20° C. Afterwards, 14 μm coronal sections were sliced on a Microm HM550 cryostat (ThermoFisher Scientific) and mounted on slides.

Immunofluorescence. For immunofluorescence (IF) of cultured samples, cells were fixed using 4% PFA for 10 minutes at 4° C., then washed 3 times with PBS. Samples were permeabilized and blocked in PBS supplemented with 0.3% Triton and 3% normal goat serum (ThermoFisher Scientific, cat. #50062Z) for 1 hour at room temperature. Primary antibodies were diluted in antibody diluent (PBS supplemented with 0.3% Triton and 1% normal goat serum) and incubated with samples for 2 hours at room temperature. Primary antibodies used include: DCX (1:500, Santa Cruz Biotechnology, cat. #sc-8066), human GFAP (1:1000, Takara, clone STEM123, cat. #Y40420), MAP2 (1:5000, Abcam, cat. #ab5392), SYN1 (1:1000, ThermoFisher Scientific, cat. #A-6442), 04 (1:500, R&D Systems, cat. #MAB1326). Samples were washed 3 times using PBS, then incubated with the appropriate secondary antibodies diluted in antibody diluent for 1 hour at room temperature (ThermoFisher Scientific). Samples were incubated in DAPI (1 μg/mL in PBS) and washed 3 times prior to imaging on a Leica DMi8 inverted microscope. For O4 staining, primary antibody was localized to the cells prior to fixation.

IF of fixed mouse brain sections was carried out in a similar manner. Samples were blocked and permeabilized, then incubated with primary antibodies diluted in antibody diluent overnight at 4° C. with agitation. Primary antibodies used include: human cytoplasmic antigen (1:1000, Takara, clone STEM121, cat. #Y40410), human GFAP (1:1000, Takara, clone STEM123, cat. #Y40420), SOX2 (1:1000, Abcam, cat. #ab97959), OLIG2 (1:500, Abcam, cat. #ab109186), MAP2 (1:5000, Abcam, cat. #ab5392), NeuN (1:1000, Abcam, cat. #ab104225).

Following incubation with secondary antibodies and DAPI, sections were floated onto slides and mounted using ProLong Gold Antifade Mountant (ThermoFisher Scientific, cat. #P36934). Imaging was done on a Leica Stellaris 8 confocal microscope. IF of fixed human fetal brain sections was carried out similarly, except in a slide mounted format. Following sectioning and mounting onto slides, Optimal Cutting Temperature OCT compound (Tissue-Tek®) was washed off with 3 changes of PBS. Tissue sections were outlined with a PAP pen (Vector Laboratories, cat. #H-4000), then stained as detailed above. Primary antibodies used include: human GFAP (1:1000, Takara, clone STEM123, cat. #Y40420), OLIG2 (1:500, Abcam, cat. #ab109186; 1:200, R&D Systems, cat. #AF2418), EGFR (1:500, Abcam, cat. #ab52894), PDGFRA (1:500, R&D Systems, cat. #AF-307), CRYAB (1:300, Abcam, cat. #ab13496), HOPX (1:1000, Sigma, cat. #HPA030180), ETV4 (ThermoFisher Scientific, 1:200, cat. #PA5-76825).

### REFERENCES

Aguirre, A., and Gallo, V. (2007). Reduced EGFR signaling in progenitor cells of the adult subventricular zone attenuates oligodendrogenesis after demyelination. Neuron Glia Biol. 3, 209-220.

Batista-Brito, R., and Fishell, G. (2013). Chapter 26—The Generation of Cortical Interneurons. In Patterning and Cell Type Specification in the Developing CNS and PNS, J.L.R. Rubenstein, and P. Rakic, eds. (Oxford: Academic Press), pp. 503-518.

Capela, A., and Temple, S. (2002). LeX/ssea-1 is expressed by adult mouse CNS stem cells, identifying them as nonependymal. Neuron 35, 865-875.

Couturier, C.P., Ayyadhury, S., Le, P.U., Nadaf, J., Monlong, J., Riva, G., Allache, R., Baig, S., Yan, X., Bourgey, M., et al. (2020). Single-cell RNA-seq reveals that glioblastoma recapitulates a normal neurodevelopmental hierarchy. Nat. Commun. 11, 3406.

Dietrich, J., Noble, M., and Mayer-Proschel, M. (2002). Characterization of A2B5+ glial precursor cells from cryopreserved human fetal brain progenitor cells. Glia 40, 65-77.

Fan, X., Fu, Y., Zhou, X., Sun, L., Yang, M., Wang, M., Chen, R., Wu, Q., Yong, J., Dong, J., et al. (2020). Single-cell transcriptome analysis reveals cell lineage specification in temporal-spatial patterns in human cortical development. Sci. Adv. 6, eaaz2978.

Fu, Y., Yang, M., Yu, H., Wang, Y., Wu, X., Yong, J., Mao, Y., Cui, Y., Fan, X., Wen, L., et al. (2021). Heterogeneity of glial progenitor cells during the neurogenesis-to-gliogenesis switch in the developing human cerebral cortex. Cell Rep. 34, 108788.

Hagemann-Jensen, M., Ziegenhain, C., Chen, P., Ramsköld, D., Hendriks, G.-J., Larsson, A.J.M., Faridani, O.R., and Sandberg, R. (2020). Single-cell RNA counting at allele and isoform resolution using Smart-seq3. Nat. Biotechnol. 38, 708-714.

Huang, W., Bhaduri, A., Velmeshev, D., Wang, S. . . . Wang, L., Rottkamp. C.A., Alvarez-Buylla, A., Rowitch, D.H., and Kriegstein, A.R. (2020). Origins and Proliferative States of Human Oligodendrocyte Precursor Cells. Cell 182, 594-608.e11.

Kriegstein, A., and Alvarez-Buylla, A. (2009). The glial nature of embryonic and adult neural stem cells. Annu. Rev. Neurosci. 32, 149-184.

Liu, Y., Zhu, H., Liu, M., Du, J., Qian, Y., Wang, Y., Ding, F., and Gu, X. (2011). Downregulation of Pax3 expression correlates with acquired GFAP expression during NSC differentiation towards astrocytes. FEBS Lett. 585, 1014-1020.

Marshall, C.A.G., Novitch, B.G., and Goldman, J.E. (2005). Olig2 Directs Astrocyte and Oligodendrocyte Formation in Postnatal Subventricular Zone Cells. J. Neurosci. 25, 7289-7298.

Nowakowski, T.J., Bhaduri, A., Pollen, A.A., Alvarado, B., Mostajo-Radji, M.A., Di Lullo, E., Haeussler, M., Sandoval-Espinosa, C., Liu, S.J., Velmeshev, D., et al. (2017). Spatiotemporal gene expression trajectories reveal developmental hierarchies of the human cortex. Science 358, 1318-1323.

Pastrana, E., Cheng, L.-C., and Doetsch, F. (2009). Simultaneous prospective purification of adult subventricular zone neural stem cells and their progeny. Proc. Natl. Acad. Sci. U. S. A. 106, 6387-6392.

Picelli, S., Faridani, O.R., Björklund, Å.K., Winberg, G., Sagasser, S., and Sandberg, R. (2014). Full-length RNA-seq from single cells using Smart-seq2. Nat. Protoc. 9, 171-181.

Polioudakis, D., de la Torre-Ubieta, L., Langerman, J., Elkins, A.G., Shi. X., Stein, J.L., Vuong, C.K., Nichterwitz, S., Gevorgian, M., Opland, C.K., et al. (2019). A Single-Cell Transcriptomic Atlas of Human Neocortical Development during Mid-gestation. Neuron 103, 785-801.e8.

Pollen, A.A., Nowakowski, T.J., Chen, J., Retallack, H., Sandoval-Espinosa, C., Nicholas, C.R., Shuga, J., Liu, S.J., Oldham, M.C., Diaz, A., et al. (2015). Molecular Identity of Human Outer Radial Glia During Cortical Development. Cell 163, 55-67.

Raff, M.C., Miller, R.H., and Noble, M. (1983). A glial progenitor cell that develops in vitro into an astrocyte or an oligodendrocyte depending on culture medium. Nature 303, 390-396.

Rash. B.G., Duque, A. . . . Morozov, Y.M., Arellano, J.I., Micali, N., and Rakic, P. (2019). Gliogenesis in the outer subventricular zone promotes enlargement and gyrification of the primate cerebrum. Proc. Natl. Acad. Sci. U. S. A. 116, 7089-7094.

Salazar, D.L., Uchida, N., Hamers, F.P.T., Cummings, B.J., and Anderson, A.J. (2010). Human neural stem cells differentiate and promote locomotor recovery in an early chronic spinal cord injury NOD-scid mouse model. PloS One 5, e12272.

Seita, J., and Weissman, I.L. (2010). Hematopoietic Stem Cell: Self-renewal versus Differentiation. Wiley Interdiscip. Rev. Syst. Biol. Med. 2, 640-653.

Stumm. R.K., Zhou, C., Ara, T., Lazarini, F., Dubois-Dalcq, M., Nagasawa, T., Höllt, V., and Schulz, S. (2003). CXCR4 regulates interneuron migration in the developing neocortex. J. Neurosci. Off. J. Soc. Neurosci. 23, 5123-5130.

Sun, Y., Goderie, S.K., and Temple, S. (2005). Asymmetric distribution of EGFR receptor during mitosis generates diverse CNS progenitor cells. Neuron 45, 873-886.

Tamaki, S.J., Jacobs, Y., Dohse, M., Capela, A., Cooper, J.D., Reitsma, M., He, D., Tushinski, R., Belichenko, P.V., Salehi, A., et al. (2009). Neuroprotection of host cells by human central nervous system stem cells in a mouse model of infantile neuronal ceroid lipofuscinosis. Cell Stem Cell 5, 310-319.

Tatsumi, K., Takebayashi, H., Manabe, T., Tanaka, K.F., Makinodan, M., Yamauchi, T., Makinodan, E., Matsuyoshi, H., Okuda, H., Ikenaka, K., et al. (2008). Genetic fate mapping of Olig2 progenitors in the injured adult cerebral cortex reveals preferential differentiation into astrocytes. J. Neurosci. Res. 86, 3494-3502.

Temple, S., and Raff, M.C. (1985). Differentiation of a bipotential glial progenitor cell in single cell microculture. Nature 313, 223-225.

Trevino, A.E., Müller, F., Andersen, J., Sundaram, L., Kathiria, A., Shcherbina, A., Farh, K., Chang, H.Y., Paşca, A.M., Kundaje, A., et al. (2021). Chromatin and gene-regulatory dynamics of the developing human cerebral cortex at single-cell resolution. Cell.

Tsukamoto, A., Uchida, N., Capela, A., Gorba, T., and Huhn, S. (2013). Clinical translation of human neural stem cells. Stem Cell Res. Ther. 4, 102.

Uchida, N., Buck, D.W., He, D., Reitsma, M.J., Masek, M., Phan, T.V., Tsukamoto, A.S., Gage, F.H., and Weissman, I.L. (2000). Direct isolation of human central nervous system stem cells. Proc. Natl. Acad. Sci. 97, 14720-14725.

Uchida, N., Chen, K., Dohse, M., Hansen, K.D., Dean, J., Buser, J.R., Riddle, A., Beardsley, D.J., Wan, Y., Gong, X., et al. (2012). Human Neural Stem Cells Induce Functional Myelination in Mice with Dysmyelination. Sci. Transl. Med. 4, Severe 10.1126/scitranslmed.3004371.

Wang, J., Sun, C., Li. J., Jiang, H., Qiu, Y., and Gong, M. (2022). Knockdown of ETV4 promotes autophagy-dependent apoptosis in GBM cells by reducing the transcriptional activation of EMP1. Oncol. Lett. 23, 41.

Windrem, M.S., Nunes, M.C., Rashbaum, W.K., Schwartz, T.H., Goodman, R.A., Mckhann, G., Roy, N.S., and Goldman, S.A. (2004). Fetal and adult human oligodendrocyte progenitor cell isolates myelinate the congenitally dysmyelinated brain. Nat. Med. 10, 93-97

Zeng, H. (2022). What is a cell type and how to define it? Cell 185, 2739-2755.

Zhang, Y., Sloan, S.A., Clarke, L.E., Caneda, C., Plaza, C.A., Blumenthal, P.D., Vogel, H., Steinberg, G.K., Edwards, M.S.B., Li, G., et al. (2016). Purification and characterization of progenitor and mature human astrocytes reveals transcriptional and functional differences with mouse. Neuron 89, 37-53.

What is claimed is:

1. A method of isolating a neural stem and progenitor cell (NSPC), the method comprising:
dissociating single cells from a brain tissue sample, contacting the singles cells with a panel of antibodies comprising one or more of a PROM1 (CD133), a CD24, a THY1 (CD90), a CXCR4, an EGFR, a PDGFRA, a CD45, a PECAM1 (CD31), a CD34, an ENG (CD105), and a GYPA (CD235a) antibody thereby producing stained single cells, and
selecting the stained single cells based on their antibody staining thereby producing an isolated NSPC.

2. The method of claim 1, wherein the brain tissue sample originates from a human.

3. The method of claim 2, wherein the brain tissue sample originates from a fetal brain.

4. The method of claim 3, wherein the brain tissue sample originates from a fetal brain that is gestational age of 16-18 weeks.

5. The method of any of the preceding claims, wherein the NSPC is a ventricular radial glia (vRG).

6. The method of any of claims 1-4, wherein the NSPC is an outer radial glia (ORG).

7. The method of any of claims 1-4, wherein the NSPC is an astrocyte (AC).

8. The method of any of claims 1-4, wherein the NSPC is a pre-oligodendrocyte precursor cell (pre-OPC).

9. The method of any of claims 1-4, wherein the NSPC is an oligodendrocyte precursor cell (OPC).

10. The method of any of claims 1-4, wherein the NSPC is an oligodendrocyte (OL).

11. The method of any of claims 1-4, wherein the NSPC is an early excitatory neuron (early ExN).

12. The method of any of claims 1-4, wherein the NSPC is a late excitatory neuron (late ExN).

13. The method of any of claims 1-4, wherein the NSPC is an inhibitory neuron (InN).

14. The method of any of claims 1-4, wherein the NSPC is a bipotent glial progenitor cell

15. The method of claim 5, wherein the vRG is defined as CD24^{-/lo} THY1^{-/lo} EGFRhi.

16. The method of claim 5 or 15, wherein the vRG is defined by expression of transcripts for one or more of SOX2, GFAP, VIM, CRYAB or FBXO32.

17. The method of claim 6, wherein the oRG is defined as CD24^{-/lo} THY1^{-/lo} EGFR−.

18. The method of claim 6 or 17, wherein the oRG is defined by expression of transcripts of one or more of SOX2, GFAP, VIM, HOPX or LIFR.

19. The method of claim 7, wherein the AC is defined as CD24^{-lo} THY1^{-/lo} EGFR+ CXCR4+.

20. The method of claim 7 or 19, wherein the AC is defined by expression of transcripts of one or more of SOX2, GFAP, VIM, PAX3 or EDNRB.

21. The method of claim 8, wherein the pre-OPC is defined as THY1^{hi} EGFR⁺ PDGFRA⁺.

22. The method of claim 8 or 21, wherein the pre-OPC is defined by expression of transcripts of one or more of OLIG1, OLIG2, SOX10, EGFR, MKI67, or PCNA.

23. The method of claim 9, wherein the OPC is defined as THY1hi EGFR⁺ PDGFRA⁺.

24. The method of claim 9 or 23, wherein the OPC is defined by expression of transcripts of one or more of OLIG1, OLIG2, SOX10, PDGFRA, or PCDH15.

25. The method of claim 10, wherein the OL is defined as THY1hi EGFR− PDGFRA−.

26. The method of claim 10 or 25, wherein the OL is defined by expression of transcripts of one or more of OLIG1, OLIG2, SOX10, MYRF, or MBP.

27. The method of claim 11, wherein the early ExN is defined as CD24+ THY1^{-/lo} CXCR4− EGFR−.

28. The method of claim 11 or 27, wherein the early ExN is defined by expression of transcripts of one or more of DCX, SOX4, SOX11, or NEUROD2.

29. The method of claim 12, wherein the late ExN is defined as CD24+ THY1^{-/lo} CXCR4− EGFR+.

30. The method of claim 12 or 29, wherein the late ExN is defined by expression of transcripts of one or more of DCX, SOX4, SOX11, or SATB2.

31. The method of claim 13, wherein the InN is defined as CD24+ THY1^{-/lo} CXCR4+ EGFR−.

32. The method of claim 13 or 31, wherein the late ExN is defined by expression of transcripts of one or more of DCX, SOX4, or SOX11.

33. The method of claim 14, wherein the BP is defined as THY1^{hi}EGFR^{hi}PDGFRA⁻.

34. The method of claim 14 or 33, wherein the late ExN is defined by expression of ETV4 (ETS Variant Transcription Factor 4).

35. The method of any of the preceding claims, wherein the dissociating comprises a combination of mechanical and enzymatic dissociation.

36. The method of any of the preceding claims, wherein the antibody panel comprises each of the PROM1 (CD133), the CD24, the THY1 (CD90), the CXCR4, an EGFR, the PDGFRA, the CD45, the PECAM1 (CD31), the CD34, the ENG (CD105), and the GYPA (CD235a) antibody.

37. The method of any of the preceding claims, wherein the antibodies are conjugated a fluorochrome.

38. The method of claim 37, wherein the selecting is performed using a fluorescence activated cell sorting.

39. The method of any of the preceding claims, wherein the isolated NSPC has a greater likelihood of producing a neurosphere relative to NSPCs isolated using other methods.

40. The method of any of the preceding claims, wherein the isolated NSPC has a 2 to 6 times improved likelihood of producing a neurosphere.

41. A method of treating an individual in need of neuron transplantation, comprising:
contacting said individual with a composition of NSPCs isolated using the methods of any of claims 1-40.

42. The method of claim 41, wherein the individual has a CNS condition.

43. The method of claim 42, wherein the CNS condition is selected from the group consisting of a neurodegenerative disease, a neuropsychiatric disorder, a channelopathy, a lysosomal storage disorder, an autoimmune disease of the CNS, a cerebral infarction, stroke, and a spinal cord injury.

44. A method of eliminating pathogenic cells in a CNS condition, the method comprising targeting specific cell surface markers of an NSPC according to any of claims 1-37 to eliminate the specific NSPC subset.

45. The method of claim 44, wherein the CNS condition is selected from the group consisting of a neurodegenerative disease, a neuropsychiatric disorder, a channelopathy, a lysosomal storage disorder, an autoimmune disease of the CNS, a cerebral infarction, stroke, and a spinal cord injury.
